(19) United States
(12) Patent Application Publication
(10) Pub. No.: US 2025/0255948 A1
(43) Pub. Date: Aug. 14, 2025

(54) PNEUMOCOCCAL CONJUGATE VACCINES AND METHODS OF USE THEREOF

(71) Applicant: Merck Sharp & Dohme LLC, Rahway, NJ (US)

(72) Inventors: Ulrike K. Buchwald, Jenkintown, PA (US); Rennie Joshi, Bryn Mawr, PA (US); Joseph G. Joyce, Lansdale, PA (US); Juliana C. Malinverni, Pennington, NJ (US); Morgan A. Monslow, Collegeville, PA (US); Adam G. C. Olmstead, Toronto (CA); Patricia Saddier, Philadelphia, PA (US); William J. Smith, Harleysville, PA (US); Priscilla T. Velentgas, South Berwick, ME (US)

(73) Assignee: Merck Sharp & Dohme LLC, Rahway, NJ (US)

(21) Appl. No.: 18/665,951

(22) Filed: May 16, 2024

Related U.S. Application Data
(60) Provisional application No. 63/553,366, filed on Feb. 14, 2024.

(51) Int. Cl. A61K 39/09; A61K 39/00; A61K 39/39; A61K 47/64; A61P 31/04

(52) U.S. Cl. CPC A61K 39/092; A61K 39/39; A61K 47/6415; A61K 47/646; A61P 31/04; A61K 2039/54; A61K 2039/545; A61K 2039/55572; A61K 2039/70

(57) ABSTRACT

The invention is related to 26-valent immunogenic compositions comprising 26 different S. pneumoniae polysaccharide carrier protein conjugates, wherein each of the conjugates comprises a polysaccharide from an S. pneumoniae serotype conjugated to a carrier protein, wherein the serotypes of S. pneumoniae are as defined herein. The 26-valent immunogenic compositions are useful for providing protection against S. pneumoniae infection and/or pneumococcal diseases caused by S. pneumoniae including pneumococcal pneumoniae, invasive pneumococcal disease and acute otitis media.

### CROSS-REFERENCE TO RELATED APPLICATIONS

This application claims the benefit of U.S. Provisional Patent Application Ser. No. 63/553,366 filed Feb. 14, 2024, the entire contents of which are incorporated by reference herein.

### FIELD OF THE INVENTION

The invention discloses pneumococcal conjugate formulations, compositions and vaccines comprising specific Streptococcus pneumoniae (S. pneumoniae) polysaccharide carrier protein conjugates. These formulations, compositions and vaccines provide an optimized global coverage against pneumococcal disease in infants and adults, balancing global S. pneumoniae serotype prevalence with potential vaccine immunogenicity loss associated with immune interference that may be present in higher valency vaccines.

### BACKGROUND

S. pneumoniae is a Gram-positive bacterium that causes invasive and non-invasive bacterial disease (such as pneumonia, bacteraemia, meningitis, and otitis media) in humans. S. pneumoniae is encapsulated by a structurally distinct capsular polysaccharide layer which confers serotype specificity. There are at least 100 known serotypes of S. pneumoniae, a subset of which are prevalent in pneumococcal disease world-wide with specific geographic variability.

Pneumococcal conjugate vaccines (PCVs) have been developed to prevent disease caused by S. pneumoniae. A 7-valent PCV (Prevnar®, Pfizer, Inc., Philadelphia, PA), containing serotypes (4, 6B, 9V, 14, 18C, 19F and 23F), was first licensed in the United States in February 2000. To date “next generation” PCVs have been developed and licensed including a 13-valent PCV (Prevnar 13®-containing serotypes 1, 3, 4, 5, 6A, 6B, 7F, 9V, 14, 18C, 19A, 19F and 23F), a 15-valent PCV (Vaxneuvance® (Merck Sharp & Dohme LLC, Rahway, NJ, USA)-containing serotypes 1, 3, 4, 5, 6A, 6B, 7F, 9V, 14, 18C, 19A, 19F, 22F, 23F and 33F)), and a 20-valent PCV (Prevnar 20® (Pfizer, Inc.)-containing serotypes 1, 3, 4, 5, 6A, 6B, 7F, 8, 9V, 10A, 11A, 12F, 14, 15B, 18C, 19A, 19F, 22F, 23F and 33F)).

A 24-valent PCV is disclosed in WO2020/131763 and contains serotypes 1, 3, 4, 5, 6A, 6B, 7F, 8, 9V, 10A, 11A, 12F, 14, 15A (15B or de-O-acetylated 15B or 15C), 18C, 19A, 19F, 22F, 23B, 23F, 24F, 33F and 35B.

Interest in higher valency PCVs is increasing and current efforts include investigational PCVs that contain 30 or more serotypes. However, as valency increases, issues associated with immune interference and decreased magnitude of serotype-specific immunogenicity are a concern. Dagan et al., Vaccine, 2010, 55:5513-5523.

Accordingly, there is a need for a PCV that balances coverage for world-wide S. pneumoniae serotype prevalence without compromising vaccine effectiveness due to immune interference potentially associated with higher valency PCVs.

### SUMMARY OF THE INVENTION

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the composition does not comprise polysaccharide carrier protein conjugates containing polysaccharides of any other S. pneumoniae serotype.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; and the carrier protein is CRM197.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; and the carrier protein is CRM197; wherein the composition does not comprise polysaccharide carrier protein conjugates containing polysaccharides of any other S. pneumoniae serotype.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; wherein the composition does not comprise polysaccharide carrier protein conjugates containing polysaccharides of any other S. pneumoniae serotype.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and the carrier protein is CRM197.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and the carrier protein is CRM197; wherein the composition does not comprise polysaccharide carrier protein conjugates containing polysaccharides of any other S. pneumoniae serotype.

The invention also provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, and the carrier protein is CRM197.

The invention also provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B, and the carrier protein is CRM197.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; and the carrier protein is CRM197; wherein the S. pneumoniae 16F polysaccharide has a molecular weight between 25 and 200 kD, the S. pneumoniae 16F polysaccharide carrier protein conjugate has a molecular weight between 1000 and 5000 kD, and the S. pneumoniae 16F polysaccharide to carrier protein ratio (w/w) 0.8 to 1.6;

and wherein the S. pneumoniae 23A polysaccharide has a molecular weight between 50 and 250 kD, the S. pneumoniae 23A polysaccharide carrier protein conjugate has a molecular weight between 1000 and 6000 kD, and the S. pneumoniae 23A polysaccharide to carrier protein ratio (w/w) 0.8 to 1.6.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; and the carrier protein is CRM197; wherein the S. pneumoniae 16F polysaccharide has a molecular weight between 50 and 150 kD, the S. pneumoniae 16F polysaccharide carrier protein conjugate has a molecular weight between 2500 and 4500 kD, and the S. pneumoniae 16F polysaccharide to carrier protein ratio (w/w) 1 to 1.4; and wherein the S. pneumoniae 23A polysaccharide has a molecular weight between 100 and 200 kD, the S. pneumoniae 23A polysaccharide carrier protein conjugate has a molecular weight between 3000 and 5500 kD, and the S. pneumoniae 16F polysaccharide to carrier protein ratio (w/w) 1 to 1.4.

In embodiments of the invention, the 26-valent immunogenic compositions described herein further comprise an adjuvant.

In other embodiments, the 26-valent immunogenic compositions described herein further comprise an adjuvant wherein the adjuvant is an aluminum phosphate adjuvant (APA).

In other embodiments, the 26-valent immunogenic compositions described herein further comprise an adjuvant wherein the adjuvant is a stable nanoemulsion (SNE) comprising sorbitan trioleate (SPAN-85), polysorbate-20 (PS-20) or polysorbate-80 (PS-80) and squalene.

In other embodiments, the 26-valent immunogenic compositions described herein further comprise an adjuvant wherein the adjuvant is a Compound B-1 stable nanoemulsion (Compound B-1-SNE) comprising Compound B-1, SPAN-85, PS-20 or PS-80 and squalene.

The 26-valent immunogenic compositions described herein are useful for the treatment and/or prevention of invasive pneumococcal disease (IPD) and/or pneumococcal pneumoniae (PP) and/or otitis media and/or acute otitis media (AOM). Thus, the invention also provides methods for the treatment and/or prevention of invasive pneumococcal disease (IPD) and/or pneumococcal pneumoniae (PP) and/or otitis media and/or acute otitis media (AOM) comprising administering any one of the 26-valent immunogenic compositions described herein to a patient in need thereof.

The 26-valent immunogenic compositions described herein may also be useful for the treatment and/or prevention of IPD and/or PP and/or otitis media and/or AOM in infants. Thus, the invention also provides methods for the treatment and/or prevention of IPD and/or PP and/or otitis media and/or AOM in infants comprising administering any one of the 26-valent immunogenic compositions described herein to a patient in need thereof, wherein the patient is an infant.

The 26-valent immunogenic compositions described herein may also be useful for the treatment and/or prevention of IPD and/or PP and/or otitis media and/or AOM in adults. Thus, the invention also provides methods for the treatment and/or prevention of IPD and/or PP and/or otitis media and/or AOM comprising administering any one of the 26-valent immunogenic compositions described herein to a patient in need thereof, wherein the patient is an adult.

Also provided are methods for inducing a protective immune response in a patient, wherein the patient is an infant or child aged 6 weeks to less than 2 years and wherein the method uses a 2+1 dosing schedule, comprising: administering a first dose of any one of the 26-valent immunogenic compositions described herein to an infant as early as 6 weeks of age, administering a second dose of the composition about 8 weeks later and administering a third (booster) dose when the patient is between 11 and 15 months of age.

The invention also provides a single 0.5 mL dose of a 26-valent immunogenic vaccine comprising: 4 μg/mL or 8 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 0.01 to 100 mg/mL SPAN-85; 0.01 to 100 mg/mL PS-20; and 0.1 to 1000 mg/mL squalene.

The invention further provides a single 0.5 mL dose of a 26-valent immunogenic vaccine comprising: 4 μg/mL or 8 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 0.1 to 10 mg/mL SPAN-85; 0.1 to 10 mg/mL PS-20; and 1 to 100 mg/mL squalene.

The invention further provides a single 0.5 mL dose of a 26-valent immunogenic vaccine comprising: 4 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 1 mg/mL SPAN-85; 1 mg/mL PS-20; and 10 mg/mL squalene.

The invention further provides a single 0.5 mL dose of a 26-valent immunogenic vaccine comprising: 4 μg/mL or 8 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 0.1 to 400 μg/mL N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide; 0.01 to 100 mg/mL SPAN-85; 0.01 to 100 mg/mL PS-20; and 0.1 to 1000 mg/mL squalene.

The invention further provides a single 0.5 mL dose of a 26-valent immunogenic vaccine comprising: 4 μg/mL or 8 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 0.1 to 40 μg/mL N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide; 0.01 to 10 mg/mL SPAN-85; 0.01 to 10 mg/mL PS-20; and 0.1 to 100 mg/mL squalene.

The invention further provides a single 0.5 mL dose of a 26-valent immunogenic vaccine comprising: 4 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 80 g/mL N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide; 5 mg/mL SPAN-85; 5 mg/mL PS-20; and 16 mg/mL squalene.

The invention further provides a single 0.5 mL dose of a 26-valent immunogenic vaccine comprising: 4 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 40 μg/mL N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide; 2.5 mg/mL SPAN-85; 2.5 mg/mL PS-20; and 8 mg/mL squalene.

The invention further provides a single 0.5 mL dose of a 26-valent immunogenic vaccine comprising: 4 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 2 μg/mL N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide; 2.5 mg/mL SPAN-85; 2.5 mg/mL PS-20; and 8 mg/mL squalene.

The invention further provides a single 0.5 mL dose of a 26-valent immunogenic vaccine comprising: 4 μg/mL of each S. pneumoniae polysaccharide, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; 0.1 μg/mL N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide; 2.5 mg/mL SPAN-85; 2.5 mg/mL PS-20; and 8 mg/mL squalene.

### BRIEF DESCRIPTION OF THE DRAWINGS

FIG. 1 shows the ratio of serotype specific IgG titers in mice following immunization with PCV26 combined with SNE compared to immunization with PCV26 combined with APA or PCV26 combined with Compound B-1-SNE compared to immunization with PCV26 combined with APA post dose 2. Serotype 6C and 15B data are included to evaluate cross-reactivity.

FIG. 2A shows mice immunized with PCV26 combined with APA, PCV26 combined with SNE, or PCV26 combined with Compound B-1-SNE are protected from S. pneumoniae 24F intratracheal challenge. Bacteremia was quantified at 24 hours post-infection with serotype 24F bacteria and presented as log CFU/mL.

FIG. 2B shows mice immunized with PCV26 combined with APA, PCV26 combined with SNE, or PCV26 combined with Compound B-1-SNE are protected from S. pneumoniae 24F intratracheal challenge. Survival was evaluated for approximately 240 hours post-infection with serotype 24F bacteria.

### DETAILED DESCRIPTION OF THE INVENTION

Global use of PCVs with increasingly broader serotype coverage has helped to reduce the burden of pneumococcal disease in infants, toddlers, children and adults. In clinical studies comparing PCVs, higher-valency PCVs have met non-inferiority criteria (based on immunoglobulin G (IgG) geometric mean concentration and IgG response rates) for most shared serotypes. A numerical trend of declining immunogenicity against shared serotypes with higher valency PCVs has also been observed; however, the clinical relevance is uncertain. Senders, S. et al., ESPID, (2023) Poster No. 1764. One hypothesis proposed to explain declining serotype-specific immunogenicity in higher valency vaccines suggests that an increase in the number of serotypes in a vaccine could lead to a reduction of the potency of individual polysaccharide conjugates due to interference between multiple polysaccharide-specific B cells competing for T-cell help from the same carrier protein. Dagan et al., Vaccine, 2010, 55:5513-5523.

While PCVs have substantially reduced disease burden caused by S. pneumoniae, serotype replacement partially negates this success due to increased disease associated with non-vaccine serotypes. S. pneumoniae serotypes 16F and 23A have emerged with increased prevalence (Kaur, R. and Pichichiro, M. JPIDS (2023) 12:135-142; Mokaya, J. et al. Microbial Genomics (2023) 9:001123; and Naucler, P. et al. Clinical Infectious Diseases (2022) 74:1338-1349).

The 26-valent PCVs disclosed herein are designed to increase S. pneumoniae serotype coverage world-wide (provide more coverage compared to licensed 13-valent, 15-valent and 20-valent PCVs) while reducing the potential risk (loss of immunogenicity) associated with immune interference that may impact PCVs currently under development that are, for example 30-valent or higher.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the composition does not comprise polysaccharide carrier protein conjugates containing polysaccharides of any other S. pneumoniae serotype.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; and wherein the carrier protein is CRM197.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of i) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; and wherein the carrier protein is CRM197; wherein the composition does not comprise polysaccharide carrier protein conjugates containing polysaccharides of any other S. pneumoniae serotype.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

The invention provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; and wherein the carrier protein is CRM197; wherein the composition does not comprise polysaccharide carrier protein conjugates containing polysaccharides of any other S. pneumoniae serotype.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and wherein the carrier protein is CRM197.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and wherein the carrier protein is CRM197; wherein the composition does not comprise polysaccharide carrier protein conjugates containing polysaccharides of any other S. pneumoniae serotype.

The invention also provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, and wherein the carrier protein is CRM197.

The invention also provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

The invention further provides a 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B, and wherein the carrier protein is CRM197.

In some embodiments, the 26-valent immunogenic compositions disclosed herein further comprise an adjuvant.

In some embodiments, the 26-valent immunogenic compositions disclosed herein further comprise an adjuvant wherein the adjuvant is APA.

In some embodiments, the 26-valent immunogenic compositions disclosed herein further comprise an adjuvant wherein the adjuvant is an SNE.

The invention provides a 26-valent immunogenic composition comprising:
i) S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of a) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, or b) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B;
ii) SPAN-85;
iii) PS-20; and
iv) squalene.

In some embodiments, the invention provides the 26-valent immunogenic compositions disclosed above wherein the concentration of SPAN-85 is 0.001 mg/mL to 10 mg/mL, the concentration of PS-20 is 0.001 mg/mL to 10 mg/mL, and the concentration of squalene is 0.01 mg/mL to 100 mg/mL.

In some embodiments, the invention provides the 26-valent immunogenic compositions disclosed above wherein the concentration of SPAN-85 is 0.005 mg/mL to 5 mg/mL, the concentration of PS-20 is 0.005 mg/mL to 5 mg/mL, and the concentration of squalene is 0.05 mg/mL to 50 mg/mL.

In some embodiments, the invention provides the 26-valent immunogenic compositions disclosed above wherein the concentration of SPAN-85 is 0.1 mg/mL to 1 mg/mL, the concentration of PS-20 is 0.1 mg/mL to 1 mg/mL, and the concentration of squalene is 1 mg/mL to 10 mg/mL.

In some embodiments, the invention provides the 26-valent immunogenic compositions disclosed above wherein the concentration of each S. pneumoniae serotype is 4 μg/mL or 8 μg/mL for a total polysaccharide concentration of 104 μg/mL or 208 μg/mL.

In some embodiments, the 26-valent immunogenic compositions disclosed herein further comprise an adjuvant wherein the adjuvant is a Compound B-1-SNE.

The invention provides a 26-valent immunogenic composition comprising:
i) S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of a) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, or b) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B;

ii) the compound: N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide (i.e., “Compound B-1”);
iii) SPAN-85;
iv) PS-20; and
v) squalene.

In some embodiments, the invention provides the 26-valent immunogenic compositions disclosed above wherein the concentration of Compound B-1 is 0.01 μg/mL to 1000 μg/mL, the concentration of SPAN-85 is 0.001 mg/mL to 30 mg/mL, the concentration of PS-20 is 0.001 mg/mL to 30 mg/mL, and the concentration of squalene is 0.01 mg/mL to 100 mg/mL.

In some embodiments, the invention provides the 26-valent immunogenic compositions disclosed above wherein the concentration of Compound B-1 is 100 μg/mL or 80 μg/mL, the concentration of SPAN-85 is 0.01 mg/mL to 10 mg/mL, the concentration of PS-20 is 0.01 mg/mL to 10 mg/mL, and the concentration of squalene is 0.03 mg/mL to 30 mg/mL.

In some embodiments, the invention provides the 26-valent immunogenic compositions disclosed above wherein the concentration of Compound B-1 is 80 μg/mL or 16 μg/mL or 4 μg/mL, the concentration of SPAN-85 is 0.2 mg/mL to 10 mg/mL, the concentration of PS-20 is 0.2 mg/mL to 10 mg/mL, and the concentration of squalene is 0.5 mg/mL to 20 mg/mL.

In some embodiments, the invention provides the 26-valent immunogenic compositions disclosed above wherein the concentration of each S. pneumoniae serotype is 4 μg/mL or 8 μg/mL for a total polysaccharide concentration of 104 μg/mL or 208 μg/mL.

The 26-valent immunogenic compositions disclosed herein are useful for the treatment and/or prevention of IPD and/or PP and/or otitis media and/or AOM.

The 26-valent immunogenic compositions disclosed herein are also useful for the treatment and/or prevention of IPD and/or PP and/or otitis media and/or AOM in infants.

The 26-valent immunogenic compositions disclosed herein are further useful for the treatment and/or prevention of IPD and/or PP and/or otitis media and/or AOM in adults.

Also provided are methods for inducing a protective immune response in patients who are infants or children aged 6 weeks to 2 years comprising a 2+1 dosing schedule wherein a first dose is administered to a patient as early as 6 weeks of age, a second dose is administered to the patient 8 weeks later and a third (booster) dose is administered when the patient is between 11 and 15 months of age.

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

The terms “aprotic solvent”, “dimethylsulfoxide (DMSO) solvent” or “DMSO conditions” when used with conjugation, such as reductive amination, refers to use of an aprotic solvent, or a combination of aprotic solvents, (or DMSO, as applicable) as the solvent for the conjugation reaction. The aprotic solvent may have some water present, for example, up to 1%, 2%, 5%, 10% or 20%.

As used herein, the term “comprising” may include the embodiments “consisting of” and “consisting essentially of.” The terms “comprise(s),” “include(s),” “having,” “has,” “may,” “contain(s),” and variants thereof are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, and excludes other components or compounds. For example, an immunogenic composition comprising 26 S. pneumoniae polysaccharide carrier protein conjugates refers to the inclusion of the 26 S. pneumoniae polysaccharide carrier protein conjugates plus optionally any other components, such as adjuvants and excipients or additional pneumococcal conjugates. The term “consisting of” or “consist(s) of” when used with specific reference to the S. pneumoniae polysaccharide carrier protein conjugates in a composition refers to a composition having the referenced S. pneumoniae polysaccharide protein conjugates and no other S. pneumoniae polysaccharide protein conjugates from a different serotype. For example, “an immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 2, 3, and 4” refers to an immunogenic composition that has S. pneumoniae polysaccharide carrier protein conjugates of S. pneumoniae serotypes 1, 2, 3 and 4 and no other S. pneumoniae serotype conjugates, but may optionally include additional components such as adjuvant(s).

An “SNE” or “stable nanoemulsion”, as used herein, refers to a composition comprising sorbitan trioleate (SPAN-85), polysorbate-20 (PS-20) and squalene, wherein the composition is in the form of a nanoemulsion. In some embodiments, an SNE comprises 0.001 mg/mL to 10 mg/mL SPAN-85, 0.001 mg/mL to 10 mg/mL PS-20 and 0.01 mg/mL to 100 mg/mL squalene. In further embodiments, an SNE comprises 0.05 mg/mL to 5 mg/mL SPAN-85, 0.05 mg/mL to 5 mg/mL PS-20 and 0.05 mg/mL to 50 mg/mL squalene. In still further embodiments, an SNE comprises 0.1 mg/mL to 1 mg/mL SPAN-85, 0.1 mg/mL to 1 mg/mL PS-20 and 1 mg/mL to 10 mg/mL squalene. As used herein, SNE is used interchangeably with “squalene emulsion”.

“Compound B-1” refers to N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide, which is a compound having the structure:

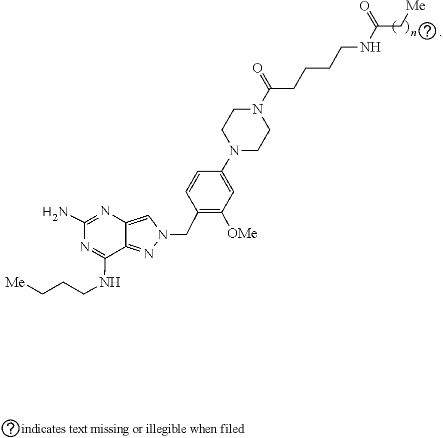 [SMILES: CCCCNc1nc(N)nc2cn(Cc3ccc(N4CCN(C(=O)CCCCNC(=O)CC)CC4)cc3OC)nc12]

A “Compound B-1-SNE” refers to a composition comprising Compound B-1, SPAN-85, PS-20 and squalene; wherein the composition is in the form of a nanoemulsion. In some embodiments, a Compound B-1 SNE comprises 0.01 μg/mL to 1000 μg/mL Compound B-1, 0.001 mg/mL to 60 mg/mL SPAN-85, 0.001 mg/mL to 60 mg/mL PS-20 and 0.01 mg/mL to 200 mg/mL squalene. In further embodiments, a Compound B-1 SNE comprises 100 μg/mL or 80 μg/mL Compound B-1, 0.01 mg/mL to 10 mg/mL SPAN-85, 0.01 mg/mL to 10 mg/mL PS-20 and 0.03 mg/mL to 30 mg/mL squalene. In still further embodiments, a Compound B-1 SNE comprises 80 μg/mL or 16 μg/mL or 4 μg/mL or 0.5 μg/mL Compound B-1, 0.2 mg/mL to 10 mg/mL SPAN-85, 0.2 mg/mL to 10 mg/mL PS-20 and 0.5 mg/mL to 20 mg/mL squalene.

“APA” means aluminum phosphate adjuvant.

“Effective amount” of a composition of the invention refers to a dose required to elicit antibodies that significantly reduce the likelihood or severity of infectivity of a microbe, e.g., S. pneumoniae, during a subsequent challenge or natural infection.

A “multivalent pneumococcal vaccine” is a pharmaceutical preparation comprising more than one active agent (e.g., pneumococcal capsular polysaccharide or pneumococcal polysaccharide protein conjugate) that provides active immunity to disease or pathological condition caused by more than one serotype of S. pneumoniae.

A “nanoemulsion”, also known as a nanometric-sized emulsion, is a fine oil-in-water (o/w) dispersion of two immiscible fluids. Nanoemulsions are a colloidal particulate system in the submicron size range acting as carriers of drug molecules. Their size varies from 10 to 1,000 nm. These carriers are solid spheres and their surface is amorphous and lipophilic.

The term “polysaccharide” is meant to include any antigenic saccharide element (or antigenic unit) commonly used in the immunologic and bacterial vaccine arts, including, but not limited to, a “saccharide”, an “oligosaccharide”, a “polysaccharide”, a “liposaccharide”, a “lipo-oligosaccharide (LOS)”, a “lipopolysaccharide (LPS)”, a “glycosylate”, a “glycoconjugate” and the like.

“PCV26” refers to an immunogenic composition containing S. pneumoniae polysaccharide (PnPs) serotypes −1, −3, −4, −5, −6A, −6B, −7F, −8, −9V, −10A, −11A, −12F, −14, −15A, (−15B or -de-O-acetylated 15B or −15C), −16F, −18C, −19A, −19F, −22F, 23A, −23B, −23F, −24F, −33F, and −35B. PCV26 is also referred to as a 26-valent immunogenic composition.

An “adjuvant,” as defined herein, is a substance that serves to enhance the immunogenicity of an immunogenic composition of the invention. An adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan.

A “patient” (alternatively referred to herein as a “subject”) refers to a mammal capable of being infected with a S. pneumoniae. In some embodiments, the patient is a human. A patient can be treated prophylactically or therapeutically. Prophylactic treatment provides sufficient protective immunity to reduce the likelihood or severity of a pneumococcal infection or the effects thereof, e.g., pneumococcal pneumonia. Therapeutic treatment can be performed to reduce the severity or prevent recurrence of a S. pneumoniae infection or the clinical effects thereof. Prophylactic treatment can be performed using a 26-valent immunogenic composition of the invention, as described herein. The compositions of the invention can be administered to the general population or to those persons at an increased risk of pneumococcal infection, e.g., infants and/or the elderly, or those who live with or care for the elderly.

Patients or subjects “in need of treatment” include those previously exposed to or infected with S. pneumoniae, those who were previously vaccinated against S. pneumoniae, as well as those prone to have an infection or any person in which a reduction in the likelihood of infection is desired, e.g., the immunocompromised, the elderly, infants, children, adults, or healthy individuals.

### II. 26-Valent Immunogenic Compositions

The invention provides 26-valent immunogenic compositions comprising 26 S. pneumoniae polysaccharide protein conjugates wherein each of the conjugates comprises a polysaccharide from an S. pneumoniae serotype conjugated to a carrier protein. The invention provides 26-valent immunogenic compositions comprising multiple S. pneumoniae polysaccharide protein conjugates wherein each of the conjugates comprises a polysaccharide from an S. pneumoniae serotype conjugated to the carrier protein CRM197. Different aspects and embodiments of the 26-valent immunogenic compositions of the invention are described, infra.

In one embodiment, the invention provides a 26-valent immunogenic composition comprising multiple S. pneumoniae polysaccharide protein conjugates, each comprising capsular polysaccharide from an S. pneumoniae serotype conjugated to a carrier protein, wherein the serotypes of S. pneumoniae consist of: i) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or ii) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or iii) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B. In another embodiment, the invention provides the 26-valent immunogenic compositions described above wherein the carrier protein is CRM197.

As used herein, de-O-acetylated serotype 15B (DeOAc15B or de-O-acetylated 15B) pneumococcal polysaccharide is substantially equivalent to serotype 15C pneumococcal polysaccharide and has a substantially identical NMR spectrum (data not shown). As used herein, the serotype de-O-acetylated 15B pneumococcal polysaccharide and the serotype 15C pneumococcal polysaccharide may each have an O-Acetyl content per repeating unit in the range of 0-5%, or in the range of 0-4%, or in the range of 0-3%, or in the range of 0-2%, or in the range of 0-1%, or in the range of 0-0.5%, or in the range of 0-0.1%, or no O-acetyl content. In a report by Spencer B. L., et al., pneumococcal polysaccharide 15C may be slightly O-acetylated (Spencer, B. L. et al., Clin. Vac. Immunol. (2017) 24 (8): 1-13). Thus, in any of the embodiments of the 26-valent immunogenic compositions herein, de-O-acetylated 15B can be used in place of serotype 15C. Processes for de-O-acetylation are known in the art, for example as described in Rajam et al., Clinical and Vaccine Immunology, 2007, 14 (9): 1223-1227.

In certain embodiments of any of the 26-valent immunogenic compositions of the invention, the composition further comprises a pharmaceutically acceptable carrier.

### Carrier Protein

In particular embodiments of the invention, CRM197 is used as the carrier protein and is well known in the art. CRM197 is a non-toxic variant (i.e., toxoid) of diphtheria toxin. The CRM197 amino acid sequence is known and is disclosed in WO2011/100151, WO2019/139692, and WO2020/131763.

In one embodiment, CRM197 is isolated from cultures of Corynebacterium diphtheria strain C7 (β197) grown in casamino acids and yeast extract-based medium. In another embodiment, CRM197 is prepared recombinantly in accordance with the methods described in U.S. Pat. No. 5,614,382. Typically, CRM197 is purified through a combination of ultra-filtration, ammonium sulfate precipitation, and ion-exchange chromatography. In some embodiments, CRM197 is prepared in Pseudomonas fluorescens using the Pelican Expression Technology®, formerly, the Pfenex Expression Technology®, (Ligand Pharmaceuticals, San Diego, CA).

Other suitable carrier proteins include additional inactivated bacterial toxins such as DT (Diphtheria toxoid) or fragment B of DT (DTFB), TT (tetanus toxoid) or fragment C of TT, pertussis toxoid, cholera toxoid (e.g., as described in WO 2004/083251).

In certain embodiments, the carrier protein is selected from the group consisting of outer membrane protein complex (OMPC), tetanus toxoid (TT), diphtheria toxoid (DT), protein D and CRM197.

In certain embodiments, the carrier protein is tetanus toxoid (TT).

In certain embodiments, the carrier protein is diphtheria toxoid (DT).

In certain embodiments, the carrier protein is CRM197.

In some embodiments of the invention, a second carrier can be used for one or more of the polysaccharide-carrier protein conjugates in the 26-valent immunogenic composition.

Further aspects of carrier proteins applicable for the 26-valent immunogenic compositions are disclosed in WO2011/100151, WO2019/139692, and WO2020/131763.

### Adjuvants

In some embodiments of the invention, the 26-valent immunogenic compositions comprise 26 different S. pneumoniae polysaccharide-carrier protein conjugates comprising capsular polysaccharide from an S. pneumoniae serotype conjugated to a carrier protein and an adjuvant, wherein the S. pneumoniae serotypes are as described herein. Suitable adjuvants to enhance effectiveness of the composition include, but are not limited to, APA, SNE and Compound B-1-SNE.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized. In one embodiment, the dose of the aluminum salt is 10, 15, 20, 25, 30, 50, 70, 100, 125, 150, 200, 300, 500, or 700 μg, or 1, 1.2, 1.5, 2, 3, 5 mg or more.

The SNE is a formulation comprising SPAN-85, PS-20 and squalene.

In some embodiments, the SNE comprises 1-34 mole % SPAN-85, 1-34 mole % PS-20 and 32-97 mole % squalene.

In some embodiments, the SNE comprises 1-7 mole % SPAN-85, 1-7 mole % PS-20 and 86-98 mole % squalene.

In some embodiments, the SNE comprises 3-4 mole % SPAN-85, 3-4 mole % PS-20 and 92-94 mole % squalene.

In some embodiments, the SNE comprises 3.98 mole % SPAN-85, 3.11 mole % PS-20 and 92.91 mole % squalene.

In some embodiments, the SNE comprises 0.15-15 mg SPAN-85, 0.15-15 mg PS-20 and 1.5-150 mg squalene.

In some embodiments, the SNE comprises 1-2 mg SPAN-85, 1-2 mg PS-20 and 5-25 mg squalene.

In some embodiments, the SNE comprises 1.5 mg SPAN-85, 1.5 mg PS-20 and 15 mg squalene.

The Compound B-1-SNE is a formulation comprising Compound B-1, SPAN-85, PS-20 and squalene.

In some embodiments, the Compound B-1-SNE comprises 0.01-0.1 mole % Compound B-1, 1.5-15.0 mole % SPAN-85, 1-10.0 mole % PS-20 and 80.0-98.0 mole % squalene.

In some embodiments, the Compound B-1-SNE comprises 0.1-1.0 mole % Compound B-1, 8.0-11.9 mole % SPAN-85, 6-10 mole % PS-20 and 78-85 mole % squalene.

In some embodiments, the Compound B-1-SNE comprises 0.01-0.1 mole % Compound B-1, 8.0-11.99 mole % SPAN-85, 6-10 mole % PS-20 and 78-85 mole % squalene.

In some embodiments, the Compound B-1-SNE comprises 200-400 μg Compound B-1, 8-16 mg SPAN-85, 8-16 mg PS-20 and 40-80 mg squalene.

In some embodiments, the Compound B-1-SNE comprises 10-20 μg Compound B-1, 8-16 mg SPAN-85, 8-16 mg PS-20 and 40-80 mg squalene.

In some embodiments, the Compound B-1-SNE comprises 0.5-3.0 μg Compound B-1, 0.6-3.6 mg SPAN-85, 0.6-3.6 mg PS-20 and 2.0-12.0 mg squalene.

In some embodiments, the Compound B-1-SNE comprises 0.5-3.0 μg Compound B-1, 0.15-0.18 mg SPAN-85, 0.15-0.18 mg PS-20 and 0.5-0.6 mg squalene.

In some embodiments, the Compound B-1-SNE comprises 0.5-3.0 μg Compound B-1, 0.15-0.95 mg SPAN-85, 0.15-0.95 mg PS-20 and 0.5-3.0 mg squalene.

In some embodiments, the Compound B-1-SNE comprises 4.0-20 μg Compound B-1, 0.24-1.3 mg SPAN-85, 0.24-1.3 mg PS-20 and 0.8-4.0 mg squalene.

In some embodiments, the Compound B-1-SNE comprises 4.0-20 μg Compound B-1, 1.2-6.5 mg SPAN-85, 1.2-6.5 mg PS-20 and 4.0-20.0 mg squalene.

In some embodiments, the Compound B-1-SNE comprises 20-100 μg Compound B-1, 1.2-6.5 mg SPAN-85, 1.2-6.5 mg PS-20 and 4.0-20.0 mg squalene.

In some embodiments, the Compound B-1-SNE comprises 20-40 μg Compound B-1, 5-8.0 mg SPAN-85, 5.0-8.0 mg PS-20 and 20.0-40.0 mg squalene.

Further aspects of adjuvants useful for the 26-valent immunogenic compositions are disclosed in WO2011/100151, WO2019/139692, and WO2020/131763.

### Formulations

The 26-valent immunogenic compositions of the invention can be formulated as single dose vials, multi-dose vials or as pre-filled glass or plastic syringes.

Formulations comprising pneumococcal conjugates are well known in the art. Further aspects of formulations applicable for the 26-valent immunogenic compositions are disclosed in WO2011/100151, WO2019/139692, and WO2020/131763.

In some embodiments, the formulation further comprises an APA. In some embodiments, the formulation further comprises histidine (20 mM), saline (150 mM) and 0.2% PS-20 at a pH of 5.8 with 250 μg (Al)/mL of APA. In one embodiment, the amount of PS-20 ranges from 0.0005% to 0.3% (w/v). In another embodiment, the amount of PS-20 ranges from 0.025% to 0.8% (w/v). In another embodiment, the amount of PS-20 ranges from 0.05% to 0.8% (w/v). In another embodiment, the amount of PS-20 ranges from 0.05% to 0.2% (w/v). In one embodiment, the process for making the formulation comprises combining a blend of 26 polysaccharide carrier protein conjugates in histidine, saline, and PS-20, then combining this blended material with APA and saline with or without antimicrobial preservatives.

In other embodiments, the formulation further comprises an SNE. In some embodiments, the formulation further comprises histidine (20 mM), saline (100 mM) and 0.075% PS-20 at a pH of 5.8, wherein the SNE comprises 12.5 mg/mL of squalene, 1.25 mg/mL of SPAN-85, and 1.25 mg/mL of PS-20. In one embodiment, the amount of PS-20 ranges from 0.0005% to 0.3% (w/v). In another embodiment, the amount of PS-20 ranges from 0.025% to 0.8% (w/v). In another embodiment, the amount of PS-20 ranges from 0.05% to 0.8% (w/v). In another embodiment, the amount of PS-20 ranges from 0.05% to 0.2% (w/v). In one embodiment, the process for making the formulation comprises combining a blend of 26 polysaccharide carrier protein conjugates in histidine, saline, and PS-20, then combining this blended material with the SNE comprising SPAN-85, PS-20 and squalene.

In other embodiments, the formulation further comprises a Compound B-1 SNE. In some embodiments, the formulation comprises histidine (13 mM), saline (75 mM) and 0.01% PS-20 at a pH of 5.8 and wherein the Compound B-1 SNE comprises 0.1 mg/mL Compound B-1, 4 mg/mL squalene, 1.2 mg/mL SPAN-85, and 1.2 mg/mL PS-20. In one embodiment, the amount of Compound B-1 ranges from 0.3 to 3% (w/w). In another embodiment, the squalene ranges from 50-98% (w/w). In another embodiment, the emulsifiers which could include PS-20 or SPAN-85 ranges from 1-30% (w/w). In one embodiment, the process for making the formulation comprises combining a blend of 26 polysaccharide carrier protein conjugates in histidine, saline, and PS-20, then combining this blended material with the Compound B-1-SNE comprising SPAN-85, PS-20 and squalene.

In particular embodiments, the 26-valent immunogenic composition of the invention comprises S. pneumoniae polysaccharide carrier protein conjugates wherein each of the conjugates comprises a polysaccharide from an S. pneumoniae serotype conjugated to a carrier protein, and wherein the composition further comprises 20-80 mM histidine pH 5.8 and 150 mM NaCl. In some embodiments, the 26-valent immunogenic composition further comprises from 0.2% to 0.8% w/v PS-20.

In some embodiments, the 26-valent immunogenic composition is prepared by individually conjugating the CRM197 carrier protein to S. pneumoniae polysaccharide (PnPs) serotypes −1, −3, −4, −5, −6A, −6B, −7F, −8, −9V, −10A, −11A, −12F, −14, −15A, (-de-O-acetylated 15B or −15B or −15C), −16F, −18C, −19A, −19F, −22F, −23A, −23B, −23F, −24F, −33F, and −35B using reductive amination in an aprotic solvent (also referred to as DMSO chemistry) and formulating in 20 mM L-Histidine pH 5.8, 150 mM NaCl and 0.1% w/v PS-20, wherein each polysaccharide serotype is at a concentration of 4 μg/mL or 8 μg/mL for a total polysaccharide concentration of 104 μg/mL or 208 μg/mL, respectively, in the formulation. In another specific embodiment, the 26-valent immunogenic composition is prepared in 20 mM L-Histidine pH 5.8, 150 mM NaCl and 0.2% w/v PS-20 at 4 μg/mL of each polysaccharide serotype for a total polysaccharide concentration of 104 μg/mL, wherein the composition further comprises 250 μg (Al)/mL in the form of APA. In another specific embodiment, the 26-valent immunogenic composition is prepared in 20 mM L-Histidine pH 5.8, 150 mM NaCl and 0.2% w/v PS-20 at 8 μg/mL of each polysaccharide serotype for a total polysaccharide concentration of 208 μg/mL, wherein the composition further comprises 250 μg (Al)/mL in the form of APA.

The amount of conjugate in each dose of the composition is selected as an amount that induces an immunoprotective response without significant, adverse effects. Such amount can vary depending upon the pneumococcal serotype. Generally, for polysaccharide-based conjugates, each dose will comprise 0.08 to 100 μg of each polysaccharide. In some embodiments of the invention, the dose of each polysaccharide conjugate is from 0.08 to 10 μg. In further embodiments, the dose of each conjugate is from 1 to 5 μg, from 0.4 to 4 μg, from 0.4 to 3 μg, from 0.4 to 2 μg, or from 0.4 to 1 μg.

In some embodiments of the compositions of the invention, all of the polysaccharide conjugates are present in the composition in the same amount. In further embodiments, the polysaccharide conjugates are present in the composition in different amounts (i.e., at least one polysaccharide conjugate is present in an amount that is different than one or more of the other polysaccharide conjugates of the composition).

Optimal amounts of components for a particular immunogenic composition can be ascertained by standard studies involving observation of appropriate immune responses in subjects. For example, in another embodiment, the dosage for human vaccination is determined by extrapolation from animal studies to human data. In another embodiment, the dosage is determined empirically.

In certain embodiments, the compositions of the invention are administered to a subject by one or more methods known to a person skilled in the art, such as parenterally, transmucosally, transdermally, intramuscularly, intravenously, intra-dermally, intra-nasally, subcutaneously, or intra-peritonealy, and formulated accordingly. In some embodiments, compositions of the invention are administered to a patient via epidermal injection, intramuscular injection, intravenous administration, intra-arterial injection, subcutaneous injection, or intra-respiratory mucosal injection of a liquid preparation. In one embodiment, the compositions of the invention are administered to a subject via parenteral administration.

Further aspects of formulations applicable for the 26-valent immunogenic compositions are disclosed in WO2011/100151, WO2019/139692, and WO2020/131763.

### III. Methods of Making

Methods of making and formulating S. pneumoniae polysaccharide carrier protein conjugates are well known in the art and are disclosed in WO2011/100151, WO2019/139692, and WO2020/131763.

Preparation and Purification of S. pneumoniae Polysaccharides

Capsular polysaccharides from S. pneumoniae can be prepared and purified by standard techniques known to those skilled in the art. The purified polysaccharides can be chemically activated to make the saccharides capable of reacting with the carrier protein.

Activation of S. pneumoniae Polysaccharides and Conjugation to Carrier Proteins

The chemical activation of the polysaccharides and subsequent conjugation to the carrier protein by reductive amination can be achieved by means described in U.S. Pat. Nos. 4,365,170, 4,673,574 and 4,902,506, U.S. Patent Application Publication Nos. 2006/0228380, 2007/184072, 2007/0231340 and 2007/0184071, and WO2006/110381, WO2008/079653, and WO2008/143709 and are well known in the art.

Purification of S. pneumoniae Polysaccharide-Carrier Protein Conjugates

After conjugation of the capsular polysaccharide to the carrier protein, the polysaccharide carrier protein conjugates are purified (enriched with respect to the amount of polysaccharide protein conjugate) by one or more of a variety of techniques. Examples of these techniques are well known to the skilled artisan and include concentration/diafiltration operations, ultrafiltration, precipitation/elution, column chromatography, and depth filtration. See, e.g., U.S. Pat. No. 6,146,902.

Formulation of S. pneumoniae Polysaccharide-Carrier Protein Conjugates

After the individual conjugates are purified, they are compounded to formulate the immunogenic compositions of the invention. These pneumococcal conjugates may be prepared by separate processes and bulk formulated into a single dosage formulation.

### IV. Methods of Use

Methods of using S. pneumoniae polysaccharide carrier protein conjugates is well know in the art and are disclosed in WO2011/100151, WO2019/139692, and WO2020/131763.

Embodiments of the invention include the 26-valent immunogenic compositions described herein (i) for use in, (ii) for use as a medicament or composition for, or (iii) for use in the preparation of a medicament for: (a) therapy (e.g., of the human body); (b) medicine; (c) inhibition of infection with Streptococcus pneumoniae; (d) induction of an immune response or a protective immune response against S. pneumoniae; (e) prophylaxis of infection by S. pneumoniae; (f) prevention of recurrence of S. pneumoniae infection; (g) reduction of the progression, onset or severity of pathological symptoms associated with S. pneumoniae infection including the prevention of associated complications such as brain damage, hearing loss, and seizures, (h) reduction of the likelihood of a S. pneumoniae infection or, (i) treatment, prophylaxis of, or delay in the onset, severity, or progression of pneumococcal disease(s), including, but not limited to: PP, pneumococcal bacteremia, pneumococcal meningitis, otitis media, AOM and sinusitis. In these uses, the multivalent pneumococcal polysaccharide-conjugate compositions of the invention can optionally be employed in combination with one or more adjuvants, or without an adjuvant.

Accordingly, the invention provides methods for the prophylactic treatment of (i.e., protection against) S. pneumoniae infection or pneumococcal disease comprising administering one or more of the 26-valent immunogenic pneumococcal polysaccharide carrier protein conjugate compositions of the invention to a patient in need of treatment.

The compositions and formulations of the invention can be used to protect or treat a human susceptible to infection, e.g., a pneumococcal infection, by means of administering such composition or formulation via a systemic or mucosal route.

In one embodiment, the invention provides a method of inducing an immune response to S. pneumoniae, comprising administering to a patient an immunologically effective amount of a 26-valent immunogenic composition of the invention. In another embodiment, the invention provides a method of vaccinating a human against a pneumococcal infection, comprising the step of administering to the human an immunologically effective amount of a 26-valent immunogenic composition of the invention.

Thus, in one aspect, the invention provides a method for (1) inducing an immune response in a human patient, (2) inducing a protective immune response in a human patient, (3) vaccinating a human patient against an infection with S. pneumoniae, or (4) reducing the likelihood of a S. pneumoniae infection in a human patient, the method comprising administering a 26-valent immunogenic composition of the invention to the patient (i.e., any 26-valent immunogenic composition described herein, such as the 26-valent immunogenic compositions described in Section II, entitled “26-Valent Immunogenic Compositions,” supra).

In one embodiment, the invention provides a method for the prevention of PP and/or IPD and/or otitis media and/or AOM in an infant (less than 1 year of age), toddler (approximately 12 to 24 months), or young child (approximately 2 to 5 years), wherein the method comprises administering to a patient in need thereof any one of the 26-valent immunogenic compositions described herein, wherein the patient is an infant, toddler, or young child.

In another embodiment, the invention provides a method for the prevention of PP and/or IPD and/or otitis media and/or AOM in a 6 week through 17 year old patient, wherein the method comprises administering to a patient in need thereof any one of the 26-valent immunogenic compositions described herein, wherein the patient is an infant or child aged 6 months to 17 years old.

In another embodiment, the invention provides a method for the prevention of PP and/or IPD and/or otitis media and/or AOM in a 6 month through 17 year old patient, wherein the method comprises administering to a patient in need thereof any one of the 26-valent immunogenic compositions described herein, wherein the patient is an infant or child aged 6 months to 17 years old.

In another embodiment, the invention provides a method for the prevention of PP and/or IPD and/or otitis media and/or AOM in adults 18 years of age and older, wherein the method comprises administering to a patient in need thereof any one of the 26-valent immunogenic compositions described herein, wherein the patient is an adult 18 years of age or older.

In another embodiment, the invention provides a method for the prevention of PP and/or IPD and/or otitis media and/or AOM in adults 50 years of age and older, wherein the method comprises administering to a patient in need thereof any one of the 26-valent immunogenic compositions described herein, wherein the patient is an adult 50 years of age or older.

In another embodiment, the invention provides a method for the prevention of PP and/or IPD and/or otitis media and/or AOM in adults 65 years of age and older, wherein the method comprises administering to a patient in need thereof any one of the 26-valent immunogenic compositions described herein, wherein the patient is an adult 65 years of age or older.

In another embodiment, the invention provides a method for the prevention of PP and/or IPD and/or otitis media and/or AOM caused by one or more of the following S. pneumoniae strains: 1, 3, 4, 5, 6A, 6B, 7F, 8, 9V, 10A, 11A, 12F, 14, 15A, 15B, 15C, 16F, 18C, 19A, 19F, 22F, 23A, 23B, 23F, 24F, 33F, and 35B, wherein the method comprises administering to a subject in need thereof any one of the 26-valent immunogenic compositions described herein.

In another embodiment of the methods above, the composition comprises 26 S. pneumoniae polysaccharide carrier protein conjugates wherein each of the conjugates comprises a polysaccharide from an S. pneumoniae serotype conjugated to a carrier protein, wherein the serotypes of S. pneumoniae consist of serotypes 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F, and 35B; or serotypes 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F, and 35B; or serotypes 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F, and 35B. In another embodiment, the carrier protein is CRM197.

The compositions of the invention are also useful in methods for providing complementary protection against S. pneumoniae in patients who had previously received a multivalent pneumococcal vaccine. In this use, the compositions of the invention can provide protection against particular S. pneumoniae serotypes that a patient had not been previously vaccinated against, can provide additional protection against S. pneumoniae serotypes that a patient had been previously vaccinated against, or can provide protection against both S. pneumoniae serotypes that a patient had not been previously vaccinated against and S. pneumoniae serotypes that a patient had been previously vaccinated against.

In embodiments of any of the methods of the invention, the patient to be treated with the compositions of the invention is a human. In certain embodiments, the human patient is an infant (approximately 6 weeks to 12 months). In certain embodiments, the human patient is a toddler (approximately 12 to 24 months), or young child (approximately 2 to 5 years). The compositions of this invention are also suitable for use with older children, adolescents and adults (e.g., aged 18 to 45 years, aged 18 to 50 years, aged 18 to 55 years, aged 18 to 60 years or 18 to 65 years). In other embodiments of any of the methods of the invention, the patient is from about 2 to about 18 years of age. In further embodiments of any of the methods of the invention, the patient is 18 years of age or older.

In further embodiments of the methods of the invention, the patient is an infant and the infant is administered 1, 2, or 3 doses of a 26-valent immunogenic composition of the invention. The amount of time between administration of each dose can vary, but an example of a dosing schedule includes administration of a dose at 2 months of age, then another administration of a dose at 4 months of age, and finally a final administration of a dose at 6 months of age. Another example of an administration schedule in infants is administration of a dose at 2 months of age and then another administration of a dose at 3 months of age. Another example of an administration schedule in infants is administration of a dose at 2 months of age and then another administration of a dose at 3 months of age, and finally a final administration of a dose at 6 months of age. In further embodiments, an infant patient can receive an additional “booster” dose of a 26-valent immunogenic composition of the invention when the infant becomes a toddler. For example, an infant is dosed at 2 months of age, then another administration of a dose at 4 months of age, and finally a final administration of a dose at 6 months of age, then, when the infant reaches the age of a toddler, an additional “booster” dose of a multivalent immunogenic composition of the invention is administered between 11 to 15 months of age.

In an embodiment, an infant is administered 1 dose of a 26-valent immunogenic composition of the invention.

In an embodiment, an infant is administered 2 doses of a 26-valent immunogenic composition of the invention.

In an embodiment, an infant is administered 3 doses of a 26-valent immunogenic composition of the invention.

In embodiments of the methods above a patient is administered multiple doses of a 26-valent immunogenic composition of the invention, wherein each dose is separated by a pre-determined amount of time. As used herein, “a pre-determined amount of time” means that each dose of the 26-valent immunogenic composition is administered approximately within a pre-selected range of time, e.g. dose 1 is administered when the patient is between 2 months and 6 months and dose 2 is administered to the patient between 3 months and 7 months, or dose 1 is administered when the patient is between 2 months and 6 months and dose 2 is administered approximately 1 month later. The pre-determined amount of time between each dose in a dosing schedule does not need to be the same between each dose; however; each dose cannot be given to the patient at the same time.

In an embodiment, a patient is administered 3 doses of a 26-valent immunogenic composition of the invention, wherein the first and second doses are administered between 2 and 10 months of age and the third dose is administered between 11 to15 months of age.

In an embodiment, a patient (an infant or a child) is administered 3 doses of a 26-valent immunogenic composition of the invention, wherein a first dose is administered to the patient as early as 6 weeks of age, a second dose is administered 8 weeks later and a third (booster) dose is administered when the patient is between 11 and 15 months of age.

In an embodiment, a patient (an infant or a child) is administered 3 doses of a 26-valent immunogenic composition of the invention, wherein a first dose is administered at 2 or 3 months of age with a second dose administered at 4 or 5 months of age and a third (booster) dose is administered at 11 or 12 months of age.

In an embodiment, a patient (an infant or a child) is administered 2 doses (a 1+1 dosing schedule) of a 26-valent immunogenic composition of the invention, wherein a first dose is administered at 12 weeks of age with a second dose (booster dose) administered at 12 months of age.

In an embodiment, a patient is administered 4 doses of a 26-valent immunogenic composition of the invention, wherein the first dose is administered at 2 months of age, the second dose is administered at 4 months of age, the third dose is administered at 6 months of age and the fourth dose is administered between 11 to 15 months of age.

In further embodiments of the methods of the invention, the human patient is elderly. In some embodiments of any of the methods of the invention, the patient is 50 years of age or older. In some embodiments of any of the methods of the invention, the patient is 55 years of age or older. In some embodiments of any of the methods of the invention, the patient is 60 years of age or older. In still further embodiments of any of the methods of the invention, the patient is 65 years of age or older. In additional embodiments of any of the methods of the invention, the patient is 70 years of age or older.

In some embodiments of any of the methods of the invention, the patient to be treated with an immunogenic composition of the invention is immunocompromised.

In some embodiments of any of the methods of the invention, the 26-valent immunogenic composition of the invention is administered concomitantly with a vaccine against influenza. In certain embodiments, the influenza vaccine is a “senior flu vaccine,” a high dose flu vaccine indicated for the elderly, e.g., persons aged 65 and older.

The invention provides a method for inducing a protective immune response in a patient against a pneumococcal infection comprising the step of administering to the patient an immunologically effective amount of any of the 26-valent immunogenic pneumococcal polysaccharide carrier protein conjugate compositions described herein. Optimal amounts of components for a particular vaccine (e.g., a 26-valent immunogenic composition of the invention) can be ascertained by standard studies involving observation of appropriate immune responses in subjects. For example, in another embodiment, the dosage for human vaccination is determined by extrapolation from animal studies to human data. In another embodiment, the dosage is determined empirically.

The methods of the invention can be used for the prevention and/or reduction of primary clinical syndromes caused by S. pneumoniae, including both invasive infections (meningitis, pneumonia, and bacteremia), and noninvasive infections (acute otitis media, and sinusitis).

Administration of the compositions of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage). In specific embodiments, the compositions of the invention are administered to the patient via intramuscular or subcutaneous administration.

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### Example 1

Preparation of S. pneumoniae Capsular Polysaccharides

Methods of culturing pneumococci are well known in the art. See, e.g., Chase, 1967, Methods of Immunology and Immunochemistry 1:52. Methods of preparing pneumococcal capsular polysaccharides are also well known in the art. See, e.g., European Patent No. EP 0 497 524 B1. The process described below generally follows the method described in European Patent No. EP 0 497 524 B1 and is generally applicable to all pneumococcal serotypes.

Isolates of pneumococcal strains for serotypes 1, 3, 4, 5, 6A, 6B, 7F, 8, 9V, 10A, 11A, 12F, 14, 15A, 15B, 16F, 18C, 19A, 19F, 22F, 23A, 23F, 33F, and 35B were obtained from Merck Culture Collection. Strains for serotype 23B were obtained from Centers of Disease Control and Prevention and University of Alabama Birmingham. Strains for serotype 24F were obtained from Merck Culture Collection and University of Alabama Birmingham. Where needed, subtypes were differentiated on the basis of Quellung reaction using specific antisera. See, e.g., U.S. Pat. No. 5,847,112. The obtained isolates were further clonally isolated by plating serially in two stages on agar plates consisting of an animal-component free medium containing soy peptone, yeast extract, and glucose without hemin. For serotype 7F, the agar plates used also contained hemin. Clonal isolates for each serotype were further expanded in liquid culture using animal-component free media containing soy peptone, yeast extract, HEPES, sodium chloride, sodium bicarbonate, potassium phosphate, glucose, and glycerol to prepare the pre-master cell banks.

The production of each serotype of pneumococcal polysaccharide consisted of a cell expansion and batch production fermentation followed by chemical inactivation prior to downstream purification. A thawed cell bank vial from each serotype was expanded using a shake flask or culture bottle containing a pre-sterilized animal-component free growth media containing soy peptone or soy peptone ultrafiltrate, yeast extract or yeast extract ultrafiltrate, HEPES, sodium chloride, sodium bicarbonate, potassium phosphate, and glucose. The cell expansion culture was grown in a sealed shake flask or bottle to minimize gas exchange with temperature and agitation control. During the cell expansion of these serotypes, temperature, pH, pressure, and agitation were controlled. Airflow overlay was also controlled as sparging was not used. After achieving a specified culture density, as measured by optical density at 600 nm, a portion of the cell expansion culture was transferred to a production fermenter containing pre-sterilized animal-component free growth media containing soy peptone or soy peptone ultrafiltrate, yeast extract or yeast extract ultrafiltrate, sodium chloride, potassium phosphate, and glucose. Temperature, pH, pressure, and agitation were controlled. Airflow overlay was also controlled as sparging was not used.

The batch fermentation was terminated via the addition of a chemical inactivating agent, phenol, when glucose was nearly exhausted. Pure phenol was added to a final concentration of 0.8-1.2% to inactivate the cells and liberate the capsular polysaccharide from the cell wall. Primary inactivation occurs for a specified time within the fermenter where temperature and agitation continue to be controlled. After primary inactivation, the batch was transferred to another vessel where it was held for an additional specified time at controlled temperature and agitation for complete inactivation. This was confirmed by either microbial plating techniques or by verification of the phenol concentration and specified time. The inactivated broth was then purified.

### Example 2

### Purification of Pneumococcal Polysaccharides

The purification process for the pneumococcal polysaccharides consisted of several centrifugation, depth filtration, concentration/diafiltration operations, and precipitation steps. All procedures were performed at room temperature unless otherwise specified.

Inactivated broth from the fermenter cultures of S. pneumoniae were flocculated with a cationic polymer (such as BPA-1000, TRETOLITE® (Baker Hughes Inc., Houston, TX), Spectrum 8160, poly(ethyleneimine), and Millipore pDADMAC). The cationic polymers binded to the impurity proteins, nucleic acids and cell debris. Following the flocculation step and an aging period, flocculated solids were removed via centrifugation and multiple depth filtration steps. Clarified broth was concentrated and diafiltered using a 100 kDa to 500 kDa MWCO (molecular weight cutoff) filter. Diafiltration was accomplished using Tris, MgCl₂ buffer and sodium phosphate buffer. Diafiltration removed residual nucleic acid and protein.

Removal of further impurities was accomplished by reprecipitation of the polysaccharide in sodium acetate and phenol with denatured alcohol and/or isopropanol. During the phenol precipitation step, sodium acetate in sodium phosphate saline buffer and phenol (liquefied phenols or solid phenols) were charged to the diafiltered retentate. Alcohol fractionation of the polysaccharide was then conducted in two stages. In the first stage a low percent alcohol was added to the preparation to precipitate cellular debris and other unwanted impurities, while the crude polysaccharide remained in solution. The impurities were removed via centrifugation followed by a depth filtration step. The polysaccharide was then recovered from the solution by adding additional isopropanol or denatured alcohol to the batch. The precipitated polysaccharide pellet was recovered by centrifugation, triturated and dried as a powder and stored frozen at −70° C.

### Example 3

### Preparation of the Serotype 1 CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular polysaccharide (Ps) powder was dissolved in water and 0.45-micron filtered. Dissolved polysaccharide was homogenized to reduce the molecular mass of the Ps. Homogenization pressure and number of passes through the homogenizer were controlled to 1000 bar/8 passes.

Size-reduced polysaccharide was concentrated and diafiltered against water using a 5 kDa nominal molecular weight cutoff (NMWCO) tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 15 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 2.0 mg Ps/mL with sucrose concentration of 10% w/v. CRM197 was formulated for lyophilization at 6 mg protein (Pr)/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.0 g Ps/L and a polysaccharide to CRM197 mass ratio of 1.6. The mass ratio was selected to control the polysaccharide to CRM197 ratio in the resulting conjugate. A mixture of sodium acetoxyborohydrides (See PCT/US2023/078423) was prepared and added (0.5 moles per mole of polysaccharide repeating unit), and conjugation proceeded for 1 hour at 22° C.

Reduction with Sodium Borohydride

Sodium borohydride (3 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 4

### Preparation of the Serotype 3 CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 500 bar/7 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 12 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 2.5 mg Ps/mL with sucrose concentration of 10% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 1.9 g Ps/L and a polysaccharide to CRM197 mass ratio of 1.5. The mass ratio was selected to control the polysaccharide to CRM197 ratio in the resulting conjugate. Sodium cyanoborohydride (0.25 mole per mole of polysaccharide repeating unit) was added, and conjugation proceeded for 1 hour at 22° C.

Reduction with Sodium Borohydride

Sodium borohydride (0.5 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered, then diluted with additional 10 mM histidine in 150 mM sodium chloride, pH 7.0 with 0.015% (w/v) PS-20, dispensed into aliquots and frozen at ≤−60° C.

### Example 5

### Preparation of the Serotype 4 CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 350 bar/7 passes.

Size-reduced polysaccharide was concentrated and diafiltered against water using a 5 kDa NMWCO tangential flow ultrafiltration membrane.

The polysaccharide solution was then adjusted to 50° C. and pH 4.1 with a sodium acetate buffer to partially deketalize the polysaccharide. The polysaccharide solution was then cooled to 22° C. prior to activation. Polysaccharide activation was initiated with the addition of a 100 mM sodium metaperiodate solution. The oxidation reaction proceeded for 6 hours at 22° C.

The activated product was diafiltered against 10 mM potassium phosphate, pH 6.4, followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 20 mM. The polysaccharide and CRM197 solutions were blended to achieve a polysaccharide concentration of 3.5 g Ps/L and a polysaccharide to CRM197 mass ratio of 2.25. The mass ratio was selected to control the polysaccharide to CRM197 ratio in the resulting conjugate. Sodium cyanoborohydride (1 mole per mole of polysaccharide repeating unit) was added, and conjugation proceeded for 8 hours at 22° C.

Reduction with Sodium Borohydride

Sodium borohydride (4 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 6

### Preparation of the Serotype 5 CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 1000 bar/8 passes.

Size-reduced polysaccharide was concentrated and diafiltered against water using a 5 kDa NMWCO tangential flow ultrafiltration membrane.

The polysaccharide solution was then adjusted to 4° C. and pH 4.1 with a sodium acetate buffer to minimize polysaccharide size reduction due to activation. Polysaccharide activation was initiated with the addition of a 100 mM sodium metaperiodate solution. The oxidation reaction proceeded for 4 hours at 4° C.

The activated product was diafiltered against 10 mM Sodium Acetate, pH 4.1, followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 2.5 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 10 mM. The polysaccharide and CRM197 solutions were blended to achieve a polysaccharide concentration of 2.0 g Ps/L and a polysaccharide to CRM197 mass ratio of 2.0. The mass ratio was selected to control the polysaccharide to CRM197 ratio in the resulting conjugate. Sodium cyanoborohydride (1 mole per mole of polysaccharide repeating unit) was added, and conjugation proceeded for 1 hour at 22° C.

Reduction with Sodium Acetoxyborohydride

A mixture of sodium acetoxyborohydrides (See PCT/US2023/078423) was prepared and added (1.0 moles per mole of polysaccharide repeating unit) following the conjugation reaction and incubated for 1 hour at 22° C. The batch was diluted into 150 mM sodium chloride, with approximately 0.025% (w/v) PS-20, at approximately 4° C. Sodium bicarbonate buffer was added to target a final concentrations of 750 mM. The batch was then concentrated and diafiltered at approximately 4° C. against 10 mM histidine in 150 mM sodium chloride pH 7.0 using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 7

### Preparation of the Serotype 6A CRM197 Conjugate

Polysaccharide was dissolved, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 200 bar/8 passes. Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.3 g Ps/L and a polysaccharide to CRM197 mass ratio of 1.5. The mass ratio was selected to control the polysaccharide to CRM197 ratio in the resulting conjugate. A mixture of sodium acetoxyborohydrides (See PCT/US2023/078423) was prepared and added (1.0 mole per mole of polysaccharide repeating unit), and conjugation proceeded for 1 hour at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) polysorbate 20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 8

### Preparation of the Serotype 6B CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 200 bar/5 passes. Size-reduced polysaccharide was concentrated and diafiltered against water using a 10 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 10 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 1.75 g Ps/L and a polysaccharide to CRM197 mass ratio of 1.35. The mass ratio was selected to control the polysaccharide to CRM197 ratio in the resulting conjugate. Sodium cyanoborohydride (1 mole per mole of polysaccharide repeating unit) was added, and conjugation proceeded for 3 hours at 22° C.

Reduction with Sodium Borohydride

Sodium borohydride (2 moles per mole of polysaccharide repeating unit) was added following the conjugation reaction and incubated for 3 hours at 22° C. The batch was diluted into 150 mM sodium chloride, with approximately 0.025% (w/v) polysorbate 20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 9

### Preparation of the Serotype 7F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 150 bar/7 passes. Size-reduced polysaccharide was concentrated and diafiltered against water using a 10 kDa NMWCO tangential flow ultrafiltration membrane.

The polysaccharide solution was then adjusted to 4° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 4° C.

The activated product was diafiltered against 10 mM potassium phosphate, pH 6.4 followed by diafiltration against water using a 10 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.04 g Ps/L and a polysaccharide to CRM197 mass ratio of 1.5. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 3 hours at 22° C. The batch was diluted into 150 mM sodium chloride, with approximately 0.025% (w/v) polysorbate 20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 10

### Preparation of the Serotype 8 CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 600 bar/6 passes. Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 4.6 g Ps/L and a polysaccharide to CRM197 mass ratio of 1.5. The mass ratio was selected to control the polysaccharide to CRM197 ratio in the resulting conjugate. After the blend, the conjugation reaction proceeded for 2 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 11

### Preparation of the Serotype 9V CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 500 bar/8 passes. Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 15 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sulfuric acid to a concentration of 2.75 mM. The polysaccharide and CRM197 solutions were blended to achieve a polysaccharide concentration of 4.2 g Ps/L and a polysaccharide to CRM197 mass ratio of 1.6. The mass ratio was selected to control the polysaccharide to CRM197 ratio in the resulting conjugate. A mixture of sodium acetoxyborohydrides (See PCT/US2023/078423) was prepared and added (1.0 mole per mole of polysaccharide repeating unit), and conjugation proceeded for 2 hours at 22° C.

Reduction with Sodium Borohydride

Sodium borohydride (3 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 12

### Preparation of the Serotype 10A CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 600 bar/5 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 3.6 g Ps/L and a polysaccharide to CRM197 mass ratio of 1.6. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 13

### Preparation of the Serotype 11A CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 800 bar/8 passes. Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.3 g Ps/L and a polysaccharide to CRM197 mass ratio of 1.5. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 14

### Preparation of the Serotype 12F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was size-reduced by acid hydrolysis by adding acetic acid to 200 mM, incubating at 80° C. for 155 minutes, then neutralizing by adding cold potassium phosphate pH 7 buffer to 400 mM.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.8 g Ps/L and a polysaccharide to CRM197 mass ratio of 1.8. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 15

### Preparation of the Serotype 14 CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 350 bar/9 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 22° C.

The activated product was diafiltered against 10 mM potassium phosphate, pH 6.4 followed by diafiltration against water using a 10 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.7 g Ps/L and a polysaccharide to CRM197 mass ratio of 1.7. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C.

Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 16

### Preparation of Serotype 15A CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 200 bar/5 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 20 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 50 mM. The polysaccharide and CRM197 solutions were blended to achieve a polysaccharide concentration of 4.0 g Ps/L and a polysaccharide to CRM197 mass ratio of 1.75. The mass ratio was selected to control the polysaccharide to CRM197 ratio in the resulting conjugate. Sodium cyanoborohydride (1 mole per mole of polysaccharide repeating unit) was added, and conjugation proceeded for 8 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 17

Preparation of Serotype 15B, or de-O-acetylated 15B, or 15C CRM197 Conjugate

Polysaccharide derived from Streptococcus pneumoniae serotype 15B was dissolved, sized to a target molecular mass, subjected to mild base hydrolysis to release O-acetyl groups, chemically activated and buffer-exchanged by ultrafiltration to yield either an activated de-O-acetylated 15B or an activated 15C polysaccharide (an activated 15B polysaccharide can be generated as described above without subjecting the 15B polysaccharide to the mild base hydrolysis). Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction, Base Hydrolysis and Oxidation

Purified serotype 15B pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 500 bar/8 passes. The size-reduced polysaccharide solution was heated to 60° C. and sodium bicarbonate pH 9.4 buffer was added to a final concentration of 50 mM. The batch was incubated with mixing for 6 hours at 60° C. to release O-acetyl groups generating the de-O-acetylated 15B and/or 15C polysaccharide (15B polysaccharide can be generated as described above without subjecting the 15B polysaccharide to heat and sodium bicarbonate). Potassium phosphate pH 6 buffer was added to a final concentration of 150 mM to neutralize pH and the solution was cooled to ambient temperature. The solution was then concentrated and diafiltered against water using a 5 kDa NMWCO tangential flow ultrafiltration membrane.

The polysaccharide solution was adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 10 mM. The polysaccharide and CRM197 solutions were blended to achieve a polysaccharide concentration of 3.2 g Ps/L and a polysaccharide to CRM197 mass ratio of 1.85. The mass ratio was selected to control the polysaccharide to CRM197 ratio in the resulting conjugate. Sodium cyanoborohydride (1 mole per mole of polysaccharide repeating unit) was added, and conjugation proceeded for 8 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 18

### Preparation of Serotype 16F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 1000 bar/8 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 20 mM. The polysaccharide and CRM197 solutions were blended to achieve a polysaccharide concentration of 2.0 g Ps/L and a polysaccharide to CRM197 mass ratio of 1.5. The mass ratio was selected to control the polysaccharide to CRM197 ratio in the resulting conjugate. Sodium cyanoborohydride (1 mole per mole of polysaccharide repeating unit) was added, and conjugation proceeded for 2 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

TABLE 1
Attributes of Serotype 16F Conjugate

| | | | Lysine | | |
|---|---|---|---|---|---|
| Oxidized | | | Consumption | Free | Free |
| Ps | Conjugate | | (mol/mol | Ps/Total | Protein/Total |
| Mn/Mw | Mn/Mw | Ps:Pr | CRM197) | Ps | Protein |
| 78/94 kD | 1890/3950 kD | 1.2 | 10.9 | <2% | <1% |

### Example 19

### Preparation of Serotype 18C CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was size-reduced by acid hydrolysis by adding acetic acid to 200 mM, incubating at 90° C. for 135 minutes, then neutralizing by adding cold potassium phosphate pH 7 buffer to 400 mM.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.5 g Ps/L and a polysaccharide to CRM197 mass ratio of 1.5. The mass ratio was selected to control the polysaccharide to CRM197 ratio in the resulting conjugate. Sodium cyanoborohydride (1 mole per mole of polysaccharide repeating unit) was added, and conjugation proceeded for 2 hours at 22° C.

Reduction with Sodium Borohydride

Sodium borohydride (2 moles per mole of polysaccharide repeating unit) was added following the conjugation reaction and incubated for 3 hours at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 20

### Preparation of Serotype 19A CRM197 Conjugate

Polysaccharide was dissolved, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Oxidation

Purified pneumococcal capsular Ps powder was dissolved in water and 0.22-micron filtered. The polysaccharide was concentrated and diafiltered against water using a 10 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 20 hours at 22° C.

The activated product was diafiltered against 10 mM potassium phosphate, pH 6.4 followed by diafiltration against water using a 10 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 3.8 g Ps/L and a polysaccharide to CRM197 mass ratio of 1.33. The mass ratio was selected to control the polysaccharide to CRM197 ratio in the resulting conjugate. Sodium cyanoborohydride (1 mole per mole of polysaccharide repeating unit) was added, and conjugation proceeded for 1.5 hours at 22° C.

Reduction with Sodium Borohydride

Sodium borohydride (2 moles per mole of polysaccharide repeating unit) was added following the conjugation reaction and incubated for 3 hours at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 21

### Preparation of Serotype 19F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 150 bar/5 passes.

Size-reduced polysaccharide was concentrated and diafiltered against water using a 10 kDa NMWCO tangential flow ultrafiltration membrane.

The polysaccharide solution was then adjusted to 4° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 4° C.

The activated product was diafiltered against 10 mM potassium phosphate, pH 6.4 followed by diafiltration against water using a 10 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.0 g Ps/L and a polysaccharide to CRM197 mass ratio of 1.2. The mass ratio was selected to control the polysaccharide to CRM197 ratio in the resulting conjugate. Sodium cyanoborohydride (1 mole per mole of polysaccharide repeating unit) was added, and conjugation proceeded for 8 hours at 22° C.

Reduction with Sodium Borohydride

Sodium borohydride (2 moles per mole of polysaccharide repeating unit) was added following the conjugation reaction and incubated for 3 hours at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane. The retentate batch was 0.2 micron filtered then incubated at 22° C. for 4.8 days.

### Final Filtration and Product Storage

The batch was then concentrated and diafiltered against 10 mM histidine in 150 mM sodium chloride, pH 7.0 at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 22

### Preparation of Serotype 22F CRM197 Conjugate

Polysaccharide was dissolved, size reduced, chemically activated and buffer-exchanged by ultrafiltration. Purified CRM197 was then conjugated to the activated polysaccharide, and the resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular polysaccharide powder was dissolved in water, and 0.45-micron filtered. Dissolved polysaccharide was homogenized to reduce the molecular mass. Homogenization pressure and number of passes through the homogenizer were controlled to 800 bar/5 passes to size-reduce to a target molecular mass. Size-reduced polysaccharide was then concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sulfuric acid to a concentration of 1.0 mM. The polysaccharide and CRM197 solutions were blended to achieve a polysaccharide concentration of 2.1 g Ps/L and a polysaccharide to CRM197 mass ratio of 1.5. The mass ratio was selected to control the polysaccharide to CRM197 ratio in the resulting conjugate. A mixture of sodium acetoxyborohydrides (See PCT/US2023/078423) was prepared and added (0.5 mole per mole of polysaccharide repeating unit), and conjugation proceeded for 2 hours at 22° C.

Reduction with Sodium Borohydride

Sodium borohydride (3 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 23

### Preparation of Serotype 23A CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass. Homogenization pressure and number of passes through the homogenizer were controlled to 1000 bar/8 passes to size-reduce to a target molecular mass. Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 20 mM. The polysaccharide and CRM197 solutions were blended to achieve a polysaccharide concentration of 2.1 g Ps/L and a polysaccharide to CRM197 mass ratio of 1.7. The mass ratio was selected to control the polysaccharide to CRM197 ratio in the resulting conjugate. Sodium cyanoborohydride (1 mole per mole of polysaccharide repeating unit) was added and conjugation proceeded for 2 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

TABLE 2
Attributes of Serotype 23A Conjugate

| | | | Lysine | | |
|---|---|---|---|---|---|
| Oxidized | | | Consumption | Free | Free |
| Ps | Conjugate | | (mol/mol | Ps/Total | Protein/Total |
| Mn/Mw | Mn/Mw | Ps:Pr | CRM197) | Ps | Protein |
| 128/145 | 3000/4800 | 1.2 | 9.7 | <2% | 3% |
| kD | kD | | | | |

### Example 24

### Preparation of Serotype 23B CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 600 bar/5 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a concentration of 20 mM. The polysaccharide and CRM197 solutions were blended to achieve a polysaccharide concentration of 5.5 g Ps/L and a polysaccharide to CRM197 mass ratio of 1.6. The mass ratio was selected to control the polysaccharide to CRM197 ratio in the resulting conjugate. Sodium cyanoborohydride (1 mole per mole of polysaccharide repeating unit) was added, and conjugation proceeded for 1 hour at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 25

### Preparation of Serotype 23F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 400 bar/5 passes.

Size-reduced polysaccharide was concentrated and diafiltered against water using a 10 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 4 hours at 22° C.

The activated product was diafiltered against 10 mM potassium phosphate, pH 6.4 followed by diafiltration against water using a 10 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.1 g Ps/L and a polysaccharide to CRM197 mass ratio of 1.25. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 3 hours at 22° C. The batch was diluted into 150 mM sodium chloride, with approximately 0.025% (w/v) PS-20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 26

### Preparation of Serotype 24F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass. Homogenization pressure and number of passes through the homogenizer were controlled to 1000 bar/8 passes to size-reduce to a target molecular mass.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 2 mg Ps/mL with sucrose concentration of 10% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide solution was spiked with sodium chloride to a final concentration of 2 mM. The polysaccharide and CRM197 solutions were blended to achieve a polysaccharide concentration of 2.0 g Ps/L and a polysaccharide to CRM197 mass ratio of 2.0. The mass ratio was selected to control the polysaccharide to CRM197 ratio in the resulting conjugate. Sodium cyanoborohydride (1 mole per mole of polysaccharide repeating unit) was added, and conjugation proceeded for 4 hours at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 27

### Preparation of Serotype 33F CRM197 Conjugate

Polysaccharide was dissolved, sized to a target molecular mass, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction and Oxidation

Purified pneumococcal capsular Ps powder was dissolved in water and 0.45-micron filtered. Dissolved polysaccharide was homogenized to reduce the molecular mass of the Ps. Homogenization pressure and number of passes through the homogenizer were controlled to 500 bar/5 passes.

Size-reduced polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 2.5 g Ps/L and a polysaccharide to CRM197 mass ratio of 1.75. The mass ratio was selected to control the polysaccharide to CRM197 ratio in the resulting conjugate. Sodium cyanoborohydride (1 mole per mole of polysaccharide repeating unit) was added, and conjugation proceeded for 1 hour at 22° C.

Reduction with Sodium Borohydride

Sodium borohydride (2 moles per mole of polysaccharide repeating unit) was added following the conjugation reaction and incubated for 1 hour at 22° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 28

### Preparation of Serotype 35B CRM197 Conjugate

Polysaccharide was dissolved, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Oxidation

Purified pneumococcal capsular Ps powder was dissolved in water and 0.45-micron filtered. Dissolved polysaccharide was concentrated and diafiltered against water using a 5 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 10 mM sodium metaperiodate solution. The oxidation reaction proceeded for 1 hour at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM₁₉₇ materials were redissolved individually in equal volumes of DMSO. The polysaccharide and CRM197 solutions were blended to achieve a polysaccharide concentration of 4.2 g Ps/L and a polysaccharide to CRM197 mass ratio of 3.0. The mass ratio was selected to control the polysaccharide to CRM197 ratio in the resulting conjugate. A mixture of sodium acetoxyborohydrides (See PCT/US2023/078423) was prepared and added (1.25 mole per mole of polysaccharide repeating unit), and conjugation proceeded for 6 hours at 22° C.

Reduction with Sodium Borohydride

Sodium borohydride (2 moles per mole of polysaccharide repeating unit) was added following the conjugation reaction and incubated for 1 hour at 34° C. The batch was diluted into 150 mM sodium chloride, with approximately 0.025% (w/v) PS-20, at approximately 4° C. Potassium phosphate buffer was then added to neutralize the pH.

### Final Filtration and Product Storage

The batch was concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. The retentate batch was 0.2 micron filtered then diluted with additional 10 mM histidine in 150 mM sodium chloride, pH 7.0 with 0.015% (w/v) PS-20, dispensed into aliquots and frozen at ≤−60° C.

### Example 29A

### Preparation of PCV26 SNE Composition

Polysaccharide carrier protein conjugates were prepared for the PCV26 immunogenicity study using methods similar to those described in prior Examples. For each serotype, polysaccharide was dissolved, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration.

As described in examples supra, several process parameters within each step, such as pH, temperature, concentration, and time were controlled to yield conjugates with desired attributes. Differences from prior Examples include: homogenization pressure and number of passes, oxidation time, polysaccharide and sucrose concentration for lyophilization, polysaccharide concentration during conjugation, polysaccharide to CRM197 mass ratio and salt concentration in the conjugation reaction.

### Preparation of the SNE Adjuvant

The SNE adjuvant formulation was prepared by combining and mixing SPAN-85, PS-20 and squalene after either dissolving in 100% EtOH together or combining as is (Table 3).

The process of making the SNE adjuvant formulation consists of 5 steps: 1) solution preparation of a component mixture of SPAN-85, PS-20 and squalene; 2) emulsion formation by means of using a controlled precipitation process via mixing systems to drive self-assembly of nanoemulsions or T-mixing; 3) ultra-filtration; 4) bioburden reduced filtration; and 5) sterile filtration and vial filling.

### Solution Preparation of Mixture

SPAN-85, PS-20 and squalene were weighed and combined before being dissolved in ethanol before being sterile filtered to form the mixture. Histidine buffer (20 mM Histidine pH 5.8) was prepared.

### SNE Formation by Means of T-Mixing

The mixture and Histidine buffer were then mixed together at adjacent ends of a T-tube mixer. The stream exiting the T-mix apparatus was immediately diluted 1:1 with 20 Histidine 0.05% PS-20 and then collected as formed SNE.

### Ultra-Filtration

The SNE intermediate was then subjected to Dialysis or ultra-filtration with a 500 kDA NMWCO in order to both concentrate the material approximately 10-fold as well as buffer exchange the material against 20 mM Histidine, 0.05% (w/v) PS-20, pH 5.8. or 20 mm Histidine pH 5.8 or 20 mM Histidine 0.05% PS-20 pH 5.8. After the dialysis or diafiltration, there was a final concentration step performed in order to achieve final target concentration.

The process of making the SNE formulation, alternatively, may consist of combining and mixing SPAN-85, PS-20 and squalene components together. Once mixed and blended, a histidine buffer is added and mixed with the initial emulsion components. Blended emulsion components are first subjected to course homogenization followed by fine homogenization to generated the SNE adjuvant bulk.

### Bioburden Reduced Filtration

The adjuvant bulk was then pre-filtered with a 0.45 μm cellulose acetate (CA) filter followed by a 0.2 μm CA bioburden-reducing filter, and stored liquid at 4° C.

### Sterile Filtration and Vial Filling

The SNE adjuvant bulk was passed through a 0.45 μm polyvinylidene fluoride (PVDF) bioburden reducing filter and a 0.22 μm PVDF sterilizing grad filter and received. The filtered adjuvant bulk was then diluted with 20 mM Histidine 0.05% (w/v) PS-20, pH 5.8 to the target SNE adjuvant concentration. This diluted final bulk adjuvant was then filled into glass vials and stored at 4° C.

TABLE 3
Composition of SNE Adjuvant

| | | Content | | Content |
|---|---|---|---|---|
| | | of Each | Molecular | of Each |
| | | Component | Weight | Component |
| Component | Description | (Mole %) | (g/mol) | (Mass %) |
| SPAN-85 | sorbitan | 88.33 | 957.5 | 33.99 |
| | trioleate | | | |
| PS-20 | polysorbate-20 | 88.33 | 1228 | 33.11 |
| Squalene | Squalene | 883.33 | 410.72 | 992.91 |
| Buffer Matrix | 20 mM | N/A | | |
| | Histidine, | | | |
| | 0.05% PS-20, | | | |
| | 75 mM NaCl, | | | |
| | pH 5.8 | | | |

### Example 29B

### Preparation of PCV26 Compound B-1-SNE Composition

Polysaccharide carrier protein conjugates were prepared for the PCV26 immunogenicity study using methods similar to those described in prior Examples. For each serotype, polysaccharide was dissolved, chemically activated and buffer-exchanged by ultrafiltration. Activated polysaccharide and purified CRM197 were individually lyophilized and redissolved in DMSO. Redissolved polysaccharide and CRM197 solutions were then combined and conjugated as described below. The resulting conjugate was purified by ultrafiltration prior to a final 0.2-micron filtration.

Several process parameters within each step, such as pH, temperature, concentration, and time were controlled to yield conjugates with desired attributes. Differences from prior Examples include: homogenization pressure and number of passes, oxidation time, polysaccharide and sucrose concentration for lyophilization, polysaccharide concentration during conjugation, polysaccharide to CRM197 mass ratio and salt concentration in the conjugation reaction.

### Preparation of Compound B-1

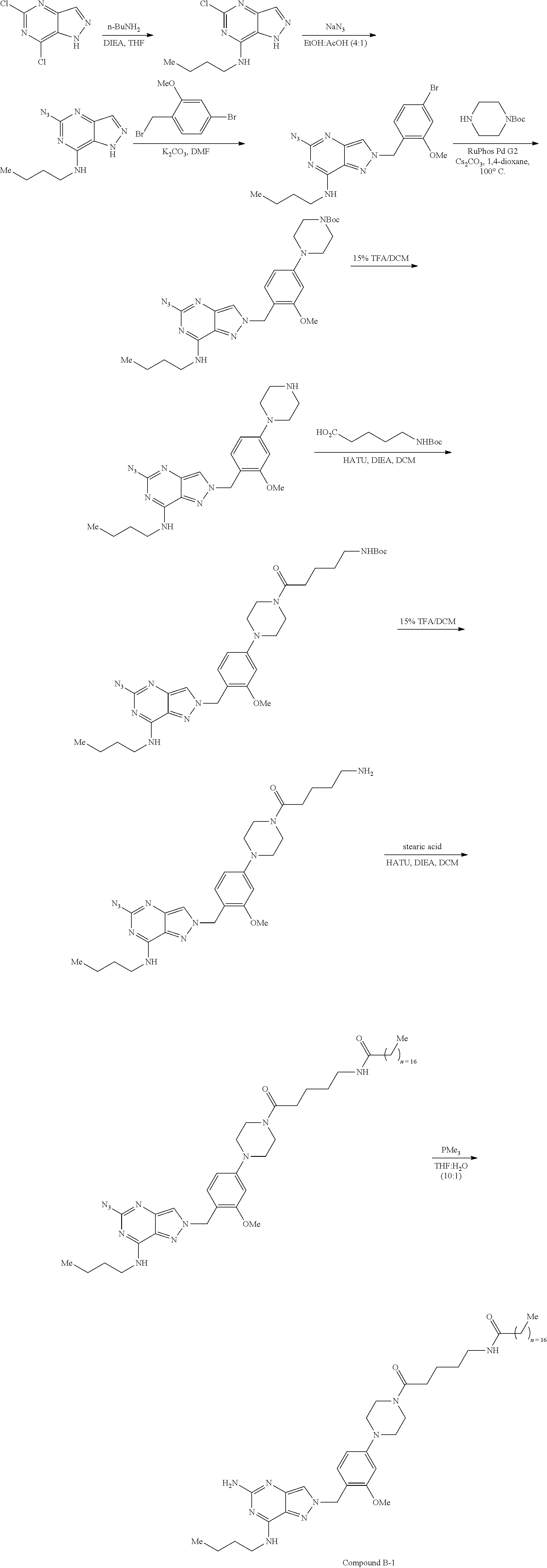

### N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide (Compound B-1)

### Step 1: N-butyl-5-chloro-1H-pyrazolo[4,3-d]pyrimidin-7-amine

To a mixture of 5,7-dichloro-1H-pyrazolo[4,3-d]pyrimidine (3 g, 15.87 mmol) in THF (30 mL) at 0° C. was added DIEA (3.08 g, 23.81 mmol) and butan-1-amine (1.741 g, 23.81 mmol). After the addition was complete, the mixture was warmed to ambient temperature and stirred for 2 h. The resulting mixture was diluted with water (200 mL), then extracted with EtOAc (3×300 mL). The combined organic extracts were washed with brine (2×300 mL), then dried (Na₂SO4), then filtered. The filtrate was concentrated to give the title compound, which was used directly in the next step without purification. MS m/z (M+H)⁺: calculated 226.1, observed 226.2.

### Step 2:5-azido-N-butyl-1H-pyrazolo[4,3-d]pyrimidin-7-amine

To a mixture of N-butyl-5-chloro-1H-pyrazolo[4,3-d]pyrimidin-7-amine (3.5 g, 15.51 mmol) in AcOH (6 mL) and EtOH (24 mL) at ambient temperature was added sodium azide (1.512 g, 23.26 mmol), then the mixture was heated to 100° C. for 3 h. The resulting mixture was cooled to ambient temperature, then diluted with water (200 mL), then extracted with EtOAc (3×300 mL). The combined organic extracts were washed with brine (2×300 mL), then dried (Na₂SO₄), then filtered, then the filtrate was concentrated. The crude product was subjected to silica gel chromatography (0-20% EtOAc/petroleum ether) to give the title compound. MS m/z (M+H)⁺: calculated 233.1, observed 233.1.

### Step 3:5-Azido-2-(4-bromo-2-methoxybenzyl)-N-butyl-2H-pyrazolo[4,3-d]pyrimidin-7-amine

5-Azido-N-butyl-1H-pyrazolo[4,3-d]pyrimidin-7-amine (938 mg, 4.04 mmol), 4-bromo-1-(bromomethyl)-2-methoxybenzene (1.36 g, 4.86 mmol), and K₂CO₃ (1.2 g, 8.68 mmol) were combined in DMF (20 mL) at ambient temperature. After stirring overnight the mixture was diluted with H₂O then extracted with EtOAc (3×). The combined organic extracts were washed with brine then dried (Na₂SO₄) then filtered then the filtrate was concentrated. The crude product was subjected to silica gel chromatography (0-50% 3:1 EtOAc:EtOH/heptane) to give the title compound. MS m/z (M+H)⁺: calculated 431.3, observed 431.0.

### Step 4: tert-butyl 4-(4-((5-azido-7-(butylamino)-2H-pyrazolo[4,3-d]pyrimidin-2-yl)methyl)-3-methoxyphenyl) piperazine-1-carboxylate

5-Azido-2-(4-bromo-2-methoxybenzyl)-N-butyl-2H-pyrazolo[4,3-d]pyrimidin-7-amine (1.38 g, 3.20 mmol), tert-butyl piperazine-1-carboxylate (0.775 g, 4.16 mmol), and Cs₂CO₃ (3.13 g, 9.60 mmol) were combined in 1,4-dioxane (20 mL). The mixture was degassed (3×pump/N₂). RuPhos Pd G2 (0.25 g, 0.322 mmol) was added then the mixture was heated to 100° C. After stirring overnight at 100° C., the mixture was cooled to ambient temperature then diluted with EtOAc then filtered through a pad of Celite®, washing with EtOAc, then the filtrate was concentrated. The crude product was subjected to silica gel chromatography (0-50% 3:1 EtOAc:EtOH/heptane) to give the title compound. MS m/z (M+H)⁺: calculated 537.3, observed 537.1.

### Step 5:5-azido-N-butyl-2-(2-methoxy-4-(piperazin-1-yl)benzyl)-2H-pyrazolo[4,3-d]pyrimidin-7-amine

To a solution of tert-butyl 4-(4-((5-azido-7-(butylamino)-2H-pyrazolo[4,3-d]pyrimidin-2-yl)methyl)-3-methoxyphenyl) piperazine-1-carboxylate (799 mg, 1.489 mmol) in DCM (8 mL) was added TFA (1.5 mL, 19.47 mmol) at ambient temperature. After 2 h the mixture was concentrated. The residue was taken up in 1:1 DCM:heptane then concentrated (2×) then dried under vacuum to give the TFA salt of the title compound which was used directly in the next step. MS m/z (M+H)⁺: calculated 437.2, observed 437.1.

### Step 6: tert-butyl (5-(4-(4-((5-azido-7-(butylamino)-2H-pyrazolo[4,3-d]pyrimidin-2-yl)methyl)-3-methoxyphenyl) piperazin-1-yl)-5-oxopentyl) carbamate

5-((tert-butoxycarbonyl)amino) pentanoic acid (421 mg, 1.936 mmol) and HATU (736 mg, 1.936 mmol) were combined in DCM (5 mL) then DIEA (1.300 ml, 7.45 mmol) was added at ambient temperature. After 1 h a solution of crude 5-azido-N-butyl-2-(2-methoxy-4-(piperazin-1-yl)benzyl)-2H-pyrazolo[4,3-d]pyrimidin-7-amine (TFA salt, 650 mg, 1.489 mmol) in DCM (10 mL) was added. After stirring overnight at ambient temperature the mixture was concentrated. The crude product was subjected to silica gel chromatography (0-100% 9:1 DCM:MeOH/DCM) to give impure product which was further purified by silica gel chromatography (0-100% 3:1 EtOAc:EtOH/heptane) to give the title compound. MS m/z (M+H)⁺: calculated 636.4, observed 636.2.

### Step 7:5-amino-1-(4-(4-((5-azido-7-(butylamino)-2H-pyrazolo[4,3-d]pyrimidin-2-yl)methyl)-3-methoxyphenyl) piperazin-1-yl) pentan-1-one

Tert-butyl (5-(4-(4-((5-azido-7-(butylamino)-2H-pyrazolo[4,3-d]pyrimidin-2-yl)methyl)-3-methoxyphenyl) piperazin-1-yl)-5-oxopentyl) carbamate (848 mg, 1.334 mmol) was taken up in DCM (10 mL) then TFA (1.1 mL, 14.28 mmol) as added at ambient temperature. After 90 minutes the mixture was concentrated give the TFA salt of the title compound which was used directly in the next step. MS m/z (M+H)⁺: calculated 536.3, observed 536.3.

### Step 8: N-(5-(4-(4-((5-azido-7-(butylamino)-2H-pyrazolo[4,3-d]pyrimidin-2-yl)methyl)-3-methoxyphenyl) piperazin-1-yl)-5-oxopentyl) stearamide

Stearic acid (493 mg, 1.734 mmol) and HATU (659 mg, 1.734 mmol) were combined in DCM (5 mL) then DIEA (1.2 ml, 6.87 mmol) was added at ambient temperature. After 30 minutes the mixture was transferred to a flask containing crude 5-amino-1-(4-(4-((5-azido-7-(butylamino)-2H-pyrazolo[4,3-d]pyrimidin-2-yl)methyl)-3-methoxyphenyl) piperazin-1-yl) pentan-1-one (TFA salt, 715 mg, 1.334 mmol) and DCM (10 mL). After stirring overnight at ambient temperature the mixture was concentrated. The crude product was subjected to silica gel chromatography (0-100% 9:1 DCM:MeOH/DCM) to give impure product which was further purified by silica gel chromatography (0-100% 3:1 EtOAc:EtOH/heptane) to give the title compound. MS m/z (M+H)⁺: calculated 802.6, observed 802.9.

### Step 9: N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide

N-(5-(4-(4-((5-azido-7-(butylamino)-2H-pyrazolo[4,3-d]pyrimidin-2-yl)methyl)-3-methoxyphenyl) piperazin-1-yl)-5-oxopentyl) stearamide (641 mg, 0.799 mmol) was taken up in THF (8 mL): H₂O (0.800 mL) resulting in a thick suspension. The mixture was heated to 50° C. and as the mixture was heating the suspension became a solution. To this was added 1M trimethylphosphine (2.4 mL, 2.400 mmol) in THF and heating continued at 50° C. After 3 h the mixture was cooled to ambient temperature then quenched with MeOH then concentrated. The crude product was subjected to silica gel chromatography (0-15% MeOH:DCM) to give the title compound. Compound B-1: MS m/z (M+H)⁺: calculated 776.6, observed 776.5. ¹H-NMR (400 MHz, DMSO-d₆) δ 11.5 (br., 1H), 8.11 (s, 1H), 7.72-7.65 (m, 2H), 7.06-6.80 (m, 1H), 6.60 (s, 1H), 6.49-6.47 (m, 1H), 6.32 (s, 1H), 5.32 (s, 2H), 3.81 (s, 3H), 3.57-3.42 (m, 6H), 3.32-3.02 (m, 4H), 2.36-2.32 (m, 2H), 2.07-1.99 (m, 4H), 1.47-1.34 (m, 8H), 1.29-1.18 (m, 30H), 0.91-0.83 (m, 6H).

### Preparation of Compound B-1-SNE Adjuvant

The Compound B-1-SNE is a multi-component emulsion formulation consisting of 3 stabilizing ingredients: SPAN-85, PS-20 and squalene and the Compound B-1 (see Table 4). This formulation is prepared by combining and mixing the Compound B-1, SPAN-85, PS-20 and squalene components after dissolving in 100% EtOH together.

The process of making the compound-stable nanoemulsion (Compound B-1-SNE) consists of 5 steps: 1) solution preparation of a component mixture that includes the 3 stabilizing ingredients: SPAN-85, PS-20 and squalene and Compound B-1; 2) SNE formation by means of T-mixing or controlled precipitation process utilizing mixing systems to drive self-assembly of the nanoemulsion; 3) ultra-filtration; 4) bioburden reduced filtration; and 5) sterile filtration and vial filling.

### Solution Preparation of Stabilizer/Compound Mixture

The stabilizer/compound components were weighed and combined before being dissolved in ethanol before being heated at 40° C. for 30 min and sterile filtered to form the component mixture.

SNE formation by means of T-mixing or controlled precipitation process utilizing mixing systems to drive self-assembly of the nanoemulsion

The component mixture and Histidine buffer were then mixed together at adjacent ends of a T-tube mixer or fluidic assembly. The stream exiting the apparatus was immediately diluted 1:1 with 20 Histidine pH 5.8, and then collected as the formed SNE.

### Ultra-Filtration

The SNE intermediate was then subjected to Dialysis or ultra-filtration with a 500 kDA NMWCO in order to both concentrate the material approximately 10-fold as well as buffer exchange the material against 20 mM Histidine, 0.05% (w/v) PS-20 and 75 mM NaCl, pH 5.8 or 20 mM Histidine pH 5.8 or 20 mM Histidine 0.05% PS-20 pH 5.8. After the dialysis or diafiltration, there was a final concentration step performed in order to achieve final target concentration.

Histidine pH 5.8, PS-20, sodium chloride, L-met, and EDTA solutions were prepared and added to a formulation vessel. The adjuvant bulk SNE was added to the formulation vessel. During the addition of the SNE to the formulation buffer, the vessel was mixed to ensure homogeneity using a magnetic sir bar or magnetic impeller. After all additions were made and the solution was stirred the formulations were filled into plastic syringes, glass syringes, or vials.

TABLE 4
Composition of Compound B-1-SNE Adjuvant

| | | Content | | Content |
|---|---|---|---|---|
| | | of Each | Molecular | of Each |
| | | Component | Weight | Component |
| Component | Description | (Mole %) | (g/mol) | (Mass %) |
| Compound | N-(5-(4-(4-((5-amino-7- | 0.01-0.1 | 431.3 | 0.30-3.03 |
| B-1 | (butylamino)-2H-pyrazolo[4,3- | | | |
| | d]pyrimidin-2-yl)methyl)-3- | | | |
| | methoxyphenyl)piperazin-1-yl)- | | | |
| | 5-oxopentyl)stearamide | | | |
| Squalene | Squalene | 80.0-98.0 | 410.72 | 61.5-97.0 |
| SPAN-85 | sorbitan trioleate | 1.5-15.0 | 957.5 | 3.1-25.0 |
| PS-20 | polysorbate-20 | 1.0-10.0 | 1228 | 1.2-24.0 |
| Buffer Matrix | 5-25 mM Histidine, pH 5.8, | N/A | | |
| | 0.05% PS-20, 0-75 mM NaCl, | | | |
| | 5-12 mM L-met, 5-10 mM | | | |
| | EDTA | | | |

### Cell-Based TLR 7/8 Assay to Evaluate Compound B-1-SNE Activity

A cell-based activation system was used to assess activity of Compound B-1-SNE, as prepared as described in examples, supra. HEK-Blue™ TLR7 and TLR8 cells (InvivoGen) were designed to assess the stimulation of TLR7 and TLR8 by monitoring the activation of NF-κB and AP-1. Compound B-1-SNE samples were diluted 1:2 across an 8-point titration in diluent buffer histidine/PS-20 in a sterile V-bottom plate. 20 μL of samples were transferred to corresponding wells of two flat bottom assay plates. Media was aspirated from HEK-Blue™ human TLR 7 and TLR 8 cells (T-75 flasks) and gently rinsed with pre-warmed (37° C.) DPBS for a total volume of 5 mL/flask. The DPBS was gently removed from the cells and cells were gently manually dislodged and resuspended in 2 mL of DPBS. Cell assay suspensions were then prepared at approximately 2.2×10⁵ cells per mL of HEK-Blue™ detection medium (InvivoGen). 180 μL of cell suspension in detection medium was added to each well of the two flat bottom assay plates containing the diluted Compound B-1-SNE samples for a final dilution of sample equaling 1:10 (180 μL of cell suspension in detection medium and 20 μL of sample). The cells were then incubated at 37° C. in 5% CO2 for 20 hrs. The reporter gene, NF-Kb, induces the production of secreted embryonic alkaline phosphatase (SEAP) when stimulated and can be monitored using the vendor supplied SEAP detection kit. SEAP colorimetric changes were read at an absorbance of 640 nm using a SpectroMax Stakmax plate reader. Compound B-1-SNE activates both TLR7 and TLR8 receptors with an EC50 of 65.4 μg/mL for TLR7 and 82.3 μg/mL for TLR8. EC50 values were calculated based on total Compound B-1-SNE concentration using GraphPad Prism Software and analyzed using agonist vs. response variable slope (four parameters) least squares fit.

### Example 30

### Formulation of PCV26

Individual pneumococcal polysaccharide carrier protein conjugates prepared as described in the Examples, supra, were used for the formulation of the 26-valent pneumococcal conjugate vaccine.

The multivalent immunogenic composition PCV26 was prepared by individually conjugating the CRM197 carrier protein to S. pneumoniae polysaccharide (PnPs) serotypes-1, −3, −4, −5, −6A, −6B, −7F, −8, −9V, −10A, −11A, −12F, −14, −15A, -de-O-acetylated 15B, −16F, −18C, −19A, −19F, −22F, −23A, −23B, −23F, −24F, −33F, and −35B using reductive amination in DMSO and formulated in 20 mM L-Histidine pH 5.8, 150 mM NaCl and 0.1% w/v PS-20 at 4 μg/mL or 8 μg/mL of each polysaccharide serotype for a total polysaccharide concentration of 104 μg/mL or 208 μg/mL, respectively, and referred to as “PCV26-no adjuvant”.

The multivalent immunogenic composition PCV26 was prepared in 20 mM L-Histidine pH 5.8, 150 mM NaCl and 0.2% w/v PS-20 at 4 μg/mL of each polysaccharide serotype for a total polysaccharide concentration of 104 μg/mL further comprising 250 μg (Al)/mL in the form of APA. This is referred to as “PCV26-APA”.

The multivalent immunogenic composition PCV26 was prepared in 20 mM L-Histidine pH 5.8, 150 mM NaCl and 0.2% w/v PS-20 at 4 μg/mL of each polysaccharide serotype for a total polysaccharide concentration of 104 μg/mL further comprising SNE adjuvant as described in the examples, supra. This is referred to as “PCV26-SNE”.

The multivalent immunogenic composition PCV26 was prepared in 20 mM L-Histidine pH 5.8, 150 mM NaCl and 0.2% w/v PS-20 at 4 μg/mL of each polysaccharide serotype for a total polysaccharide concentration of 104 μg/mL further comprising Compound B-1-SNE adjuvant as described in the examples, supra. This is referred to as “PCV26-Compound B-1-SNE”.

The required volume of bulk conjugates needed to obtain the target concentration of individual serotypes were calculated based on batch volume and concentration of individual bulk polysaccharide concentrations. The individual conjugates were added to a solution of histidine, sodium chloride and PS-20 to produce a 2×-4× conjugate blend. The formulation vessel containing the conjugate blend is mixed using a magnetic stir bar, sterile filtered into another vessel. The sterile filtered 2×-4× blend is either added to another vessel containing APA or SNE or diluted with saline to achieve the desired target total polysaccharide, excipient, APA and SNE (if required) concentrations. The formulations are then filled into glass vials or syringes and stored at 2-8° C.

### Example 31

### PCV26 Immunogenicity and Functional Antibody Responses in Mice

Female Balb/c mice (6-8 weeks old, n=15/group) were intramuscularly immunized with 0.1 mL of 26-valent pneumococcal conjugate vaccine (PCV) on day 0, day 28, and day 56. PCV26 was administered at 0.4 μg of each pneumococcal polysaccharide (1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F, 35B) all individually conjugated to CRM197 and adjuvanted with aluminum phosphate adjuvant (APA), stable nanoemulsion (SNE), or Compound B-1-SNE. Adjuvants were utilized at concentrations as described in Table 5. Mice were observed at least daily by trained animal care staff for any signs of illness or distress.

TABLE 5
PCV26 Formulations
Formulation

PCV26/APA (104 μg (PnPs)/mL) in 18.1 mM L-histidine, 150 mM NaCl,
0.09% w/v PS-20, pH 5.8 250 μg/mL APA
PCV26/SNE (104 μg (PnPs)/mL) in 16.3 mM L-histidine, 98.5 mM NaCl,
0.07% w/v PS-20, pH 5.8 15 mg/mL SNE (12.5 mg/mL
of squalene; 1.25 mg/mL of SPAN-85; and 1.25 mg/mL of PS-20)
PCV26/Compound B-1-SNE (104 μg (PnPs)/mL) in 13.3 mM L-histidine,
75 mM NaCl, 0.09% w/v PS-20, pH 5.8 6.5 mg/mL Compound B-1-SNE
(0.1 mg/mL of Compound B-1, 1.2 mg/mL of SPAN-85; 1.2
mg/mL of PS-20; and 4 mg/mL of squalene)

Mouse sera were collected prior to study start (pre-immune, day 0) and on days 14 (PD1), 42 (PD2), and 70 (PD3). Sera were evaluated for IgG immunogenicity using a multiplexed electrochemiluminescence (ECL) assay. This assay was developed for use with mouse serum based on the human assay described by Marchese et al. (Marchese R. D., et al., Clin. Vaccine Immunol. (2009) 16 (3): 387-396) using technology developed by MesoScale Discovery (a division of MesoScale Diagnostics, LLC, Gaithersburg, MD) which utilizes a SULFO-TAG™ label that emits light upon electrochemical stimulation. SULFO-TAG™-labeled anti-mouse IgG was used as the secondary antibody for testing mouse serum samples. Functional antibody titers weredetermined through multiplexed opsonophagocytic assays (MOPA) based on previously described protocols at www.vaccine.uab.edu and Opsotiter® 3 software owned by and licensed from University of Alabama (UAB) Research Foundation (Caro-Aguilar, I. et al., Vaccine (2017) 35 (6): 865-872; and Burton R. L., and Nahm M. H., Clin. Vaccine Immunol. (2006) 19 (9): 1004-1009).

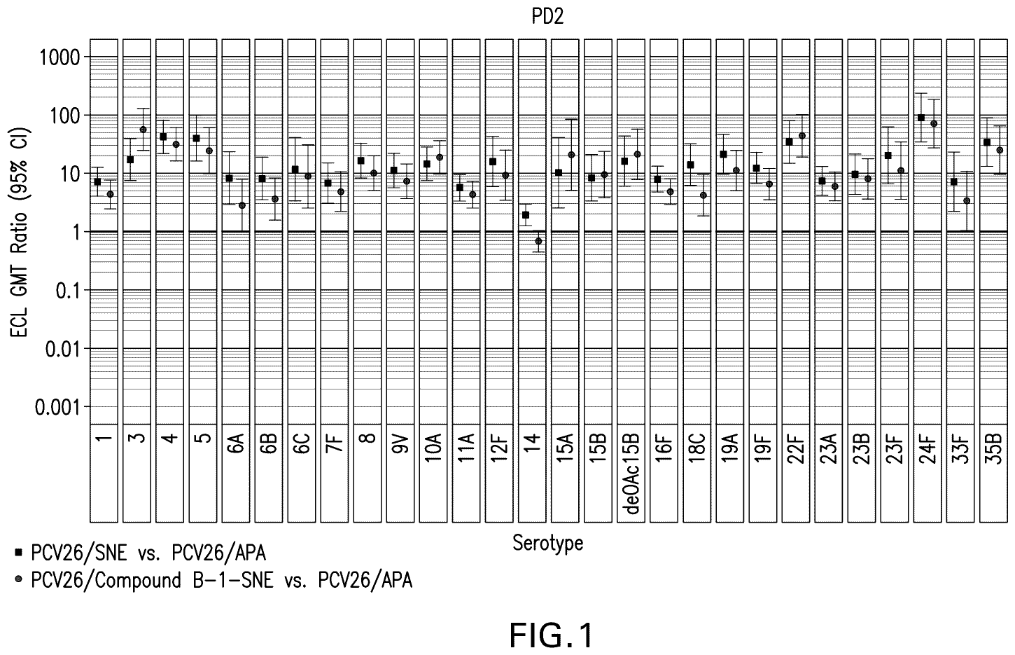

Mouse sera were tested individually in the ECL assay to determine antibody titers and generated antibody titers for all serotypes in the vaccine. Day 0 sera was pooled by group prior to testing in the ECL assay. Antibody titers in mice immunized with PCV26/SNE or PCV26/Compound B-1-SNE were comparable to or higher than titers in mice immunized with PCV26/APA and the maximal difference was observed by day 42 (FIG. 1).

It is also of note that PCV26, which contains polysaccharide conjugates 15A-CRM197, deOAc15B-CRM197, 6A-CRM197, and 6B-CRM197 also provided cross-reactivity to 15B and 6C and these titers in mice immunized with PCV26/SNE or PCV26/Compound B-1-SNE were comparable to or higher than those in mice immunized with PCV26/APA (FIG. 1).

For all timepoints, mouse sera was pooled by group prior to testing in the MOPA to determine functional antibody titers and PCV26 generated functional antibody titers in mice which killed vaccine-type bacterial serotypes. Similar to results from the ECL assay, functional antibody titers in mice immunized with PCV26/SNE or PCV26/Compound B-1-SNE were comparable to or trended higher than titers in mice immunized with PCV26/APA and the maximal difference was observed by day 42 (PD2).

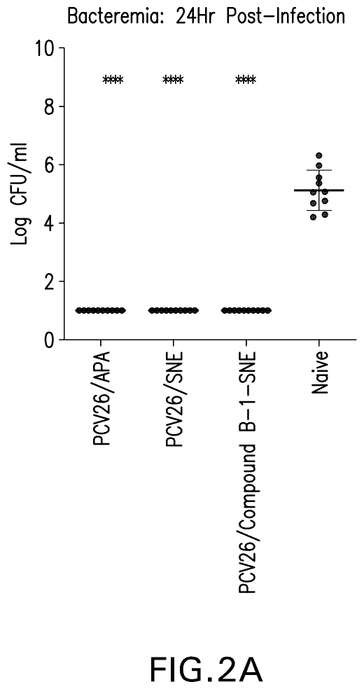

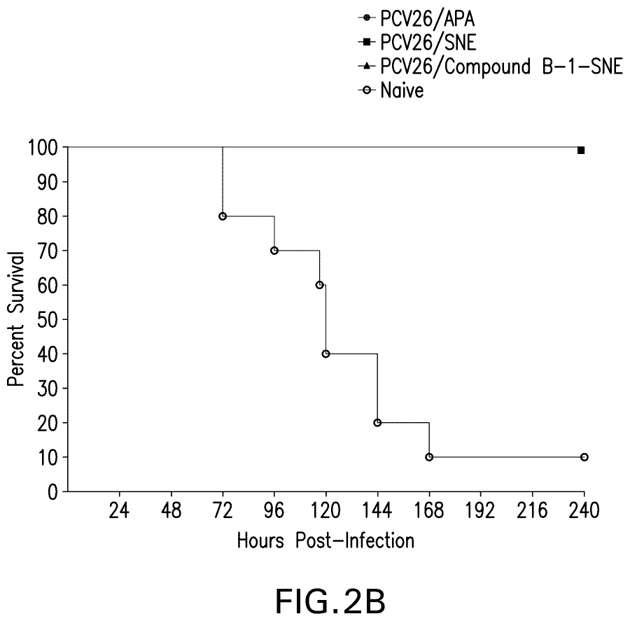

On day 77, 10 mice per group were intratracheally challenged with 10⁵ cfu of S. pneumoniae serotype 24F bacteria in 0.1 mL of PBS. After challenge, mice were weighed daily and blood was collected at 24 hours, 48 hours and 72 hours to assess bacteremia. Mice were monitored by trained animal care staff for any sign of illness or distress in accordance with a schedule approved by the Institutional Animal Care and Use Committee and euthanized if defined criterion were met. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc. All PCV26 immunized mice were protected from bacteremia and showed 100% survival after intratracheal challenge with S. pneumoniae serotype 24F. One-Way ANOVA with Dunnett Multiple comparisons test indicated that PCV26/APA, PCV26/SNE, and PCV26/Compound B-1-SNE immunized groups were significantly protected from bacteremia when compared to the naïve control group (P<0.0001) (FIG. 2A). Mantel-Cox log-rank test of survival curves indicated that PCV26/APA, PCV26/SNE, and PCV26/Compound B-1-SNE immunized groups were significantly protected from challenge when compared to the naïve control group (P<0.0001) (FIG. 2B).

What is claimed is:

1. A 26-valent immunogenic composition comprising S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B; or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

2. The 26-valent immunogenic composition of claim 1, wherein the carrier protein is CRM197.

3. The 26-valent immunogenic composition of claim 2, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

4. The 26-valent immunogenic composition of claim 1, wherein the composition further comprises an adjuvant.

5. The 26-valent immunogenic composition of claim 4, wherein the adjuvant is an aluminum phosphate adjuvant (APA).

6. The 26-valent immunogenic composition of claim 4, wherein the adjuvant comprises i) sorbitan trioleate (SPAN-85); ii) polysorbate-20 (PS-20) or polysorbate-80 (PS-80); and iii) squalene.

7. The 26-valent immunogenic composition of claim 6, wherein the composition comprises PS-20.

8. The 26-valent immunogenic composition of claim 7 wherein the concentration of SPAN-85 is 0.001 mg/mL to 100 mg/mL, the concentration of PS-20 is 0.001 mg/mL to 100 mg/mL, and the concentration of squalene is 0.01 mg/mL to 100 mg/mL.

9. The 26-valent immunogenic composition of claim 7 wherein the concentration of SPAN-85 is 0.01 mg/mL to 50 mg/mL, the concentration of PS-20 is 0.01 mg/mL to 50 mg/mL, and the concentration of squalene is 0.02 mg/mL to 20 mg/mL.

10. The 26-valent immunogenic composition of claim 7 wherein the concentration of SPAN-85 is 0.1 mg/mL to 10 mg/mL, the concentration of PS-20 is 0.1 mg/mL to 10 mg/mL, and the concentration of squalene is 1 mg/mL to 20 mg/mL.

11. The 26-valent immunogenic composition of claim 4, wherein the adjuvant comprises i) N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide, or a pharmaceutically acceptable salt thereof; ii) sorbitan trioleate (SPAN-85); iii) polysorbate-20 (PS-20); and iv) squalene.

12. A 26-valent immunogenic composition comprising: i) S. pneumoniae polysaccharide carrier protein conjugates, wherein each of the conjugates comprises a polysaccharide of a particular S. pneumoniae serotype conjugated to a carrier protein, wherein the S. pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B; and the carrier protein is CRM197; ii) N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide, or a pharmaceutically acceptable salt thereof; iii) sorbitan trioleate (SPAN-85); iv) polysorbate-20 (PS-20); and v) squalene.

13. The 26-valent immunogenic composition of claim 12 wherein the concentration of N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide, or a pharmaceutically acceptable salt thereof, or pharmaceutically acceptable salt thereof is 0.01 μg/mL to 1000 μg/mL, the concentration of SPAN-85 is 0.01 mg/mL to 50 mg/mL, the concentration of PS-20 is 0.01 mg/mL to 50 mg/mL, and the concentration of squalene is 0.02 mg/mL to 20 mg/mL.

14. The 26-valent immunogenic composition of claim 12 wherein the concentration of N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide, or a pharmaceutically acceptable salt thereof, or pharmaceutically acceptable salt thereof is 0.1 μg/mL to 100 μg/mL, the concentration of SPAN-85 is 0.01 mg/mL to 50 mg/mL, the concentration of PS-20 is 0.01 mg/mL to 50 mg/mL, and the concentration of squalene is 0.02 mg/mL to 20 mg/mL.

15. The 26-valent immunogenic composition of claim 12 wherein the concentration of N-(5-(4-(4-((5-amino-7-(butylamino)-2H-pyrazolo[4,3-d]pyrimidin-2-yl)methyl)-3-methoxyphenyl) piperazin-1-yl)-5-oxopentyl) stearamide, or a pharmaceutically acceptable salt thereof, or pharmaceutically acceptable salt thereof is 80 μg/mL or 16 μg/mL or 4 μg/mL, the concentration of SPAN-85 is 0.1 mg/mL to 10 mg/mL, the concentration of PS-20 is 0.1 mg/mL to 10 mg/mL, and the concentration of squalene is 1 mg/mL to 20 mg/mL.

16. A method for inducing an immune response against S. pneumoniae in a patient in need thereof comprising administering the 26-valent immunogenic composition of claim 1 to the patient.

17. A method for inducing a protective immune response against S. pneumoniae in a patient in need thereof comprising administering the 26-valent immunogenic composition of claim 1 to the patient.

18. The method of claim 17, wherein the patient is an infant or a toddler.

19. The method of claim 17, wherein the patient is an adult.

20. A method for the prevention of invasive pneumococcal disease (IPD) and/or pneumococcal pneumoniae (PP) in a patient in need thereof comprising administering the 26-valent immunogenic composition of claim 1 to the patient.

21. The method of claim 20, wherein the patient is an infant or a toddler.

22. The method of claim 20, wherein the patient is an adult.

23. A method of prevention of otitis media and/or acute otitis media (AOM) in a patient in need thereof comprising administering the 26-valent immunogenic composition of claim 1 to the patient.

24. The method of claim 23, wherein the patient is an infant or a toddler.

25. The method of claim 23, wherein the patient is an adult.

26. The method of claim 20 wherein the 26-valent immunogenic composition is administered by intramuscular injection.

27. The method of claim 20, wherein the patient is administered two or more doses of the 26-valent immunogenic composition.

28. The method of claim 27, wherein each dose is separated by a pre-determined amount of time.

29. The method of claim 21, wherein the patient is administered 3 doses of the 26-valent immunogenic composition; wherein the first and second doses are administered when the patient is between 2 and 10 months of age and the third dose is administered when the patient is between 11 and 15 months of age.

30. The method of claim 21, wherein the patient is administered 3 doses of the 26-valent immunogenic composition; wherein the first dose is administered when the patient is 6 weeks of age or younger, the second dose is administered 8 weeks after the first dose and the third dose is administered when the patient is between 11 and 15 months of age.

31. The method of claim 21, wherein the patient is administered 3 doses of the 26-valent immunogenic composition; wherein the first dose is administered when the patient is 2-3 months of age, the second dose is administered when the patient is 4-5 months of age and the third dose is administered when the patient is 11-12 months of age.

32. The method of claim 20, wherein the 26-valent immunogenic composition is administered by intramuscular injection.
